(19) United States
(12) Patent Application Publication
(10) Pub. No.: US 2026/0157687 A1
(43) Pub. Date: Jun. 11, 2026

(54) MULTIMODAL ANALYSIS USING VOCAL BIOMARKERS FOR HEALTH CONDITIONS

(71) Applicant: Canary Speech, Inc., Provo, UT (US)

(72) Inventors: Henry O'Connell, Spanish Fork, UT (US); Nate Blaylock, Forney, TX (US)

(73) Assignee: Canary Speech, Inc., Provo, UT (US)

(21) Appl. No.: 19/323,735

(22) Filed: Sep. 9, 2025

Related U.S. Application Data
(60) Provisional application No. 63/714,127, filed on Oct. 30, 2024.

(51) Int. Cl. A61B 5/00

(52) U.S. Cl. CPC A61B 5/4803; A61B 5/7221; A61B 5/7267; A61B 5/7271

(57) ABSTRACT

Disclosed herein are techniques for diagnosing health conditions of a subject based on the subject's vocal biomarkers and health records, including techniques for generating one or more models (e.g., machine learning models) trained to predict a health condition in a subject. One or more techniques may include receiving speech data and health record data, analyzing the received speech data and health record data using one or more trained models, and outputting the prediction of whether the subject has any of the one or more health conditions.

### RELATED APPLICATIONS

This application claims the benefit under 35 U.S.C. § 119 (e) of U.S. Provisional Application Ser. No. 63/714,127, titled “MULTIMODAL ANALYSIS USING VOCAL BIOMARKERS AND FOUNDATION MODELS FOR HEALTH CONDITIONS,” filed on Oct. 30, 2024, which is incorporated by reference herein its entirety.

Some embodiments described herein may incorporate or leverage some of the subject matter of U.S. Pat. Nos. 10,152,988, 10,311,980, and/or U.S. Patent Application No. 12,125,497, each of which are incorporated by reference herein in their entirety and at least for their discussions of identification and extraction of vocal biomarkers, training/configurating detectors of health conditions based on vocal biomarkers, and detection of health conditions based on vocal biomarkers.

### BACKGROUND

To receive an accurate diagnosis, subjects may undergo a comprehensive evaluation process that is often facilitated by a primary care physician or generalist, who assesses the subject's symptoms and determines the need for specialized care. If necessary, the subject is then referred to a specialist, such as a cardiologist, oncologist, or neurologist, who has advanced training and expertise in a specific area of medicine. The specialist may conduct further evaluation and testing to confirm or rule out a diagnosis.

### SUMMARY

In one embodiment, there is provided a method for predicting whether a subject has one or more health conditions. The method includes determining a prediction by analyzing audio data that includes a plurality of speech samples of the subject and analyzing health record data. The prediction is determined at least in part by analyzing the audio data and the health record data using one or more trained models that were trained with prior audio data and prior health record data of a plurality of prior subjects along with corresponding information regarding whether each prior subject had one or more health conditions. The method further includes outputting the prediction of whether the subject has any of the health conditions.

In another embodiment, there is provided a computer-readable storage medium storing computer-executable instructions that, when executed by a processor, cause the processor to carry out a method. The method includes determining a prediction by analyzing audio data that includes a plurality of speech samples of the subject and analyzing health record data. The prediction is determined at least in part by analyzing the audio data and the health record data using one or more trained models that were trained with prior audio data and prior health record data of a plurality of prior subjects along with corresponding information regarding whether each prior subject had one or more health conditions. The method further includes outputting the prediction of whether the subject has any of the health conditions.

In yet another embodiment, there is provided an apparatus comprising a processor and a storage medium having stored computer-executable instructions. When executed by the processor, the instructions cause the processor to perform a method. The method includes determining a prediction by analyzing audio data that includes a plurality of speech samples of the subject and analyzing health record data. The prediction is determined at least in part by analyzing the audio data and the health record data using one or more trained models that were trained with prior audio data and prior health record data of a plurality of prior subjects along with corresponding information regarding whether each prior subject had one or more health conditions. The method further includes outputting the prediction of whether the subject has any of the health conditions.

The foregoing is a non-limiting summary of the invention, which is defined by the attached claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a block diagram of a system with which one or more embodiments may operate;

FIG. 2 is an example audio processing facility and health record processing facility, in accordance with one or more embodiments;

FIG. 3 is an example system that may be used to select features for training a machine learning model for diagnosing a health condition based on audio data and health record data and then using the selected features to train the machine learning model;

FIG. 4 is a flowchart of a process that may be implemented in one or more embodiments to evaluate audio data and health record data for identifying features related to a health condition and generating a result that may be provided to a clinician to assist or inform the clinician's diagnosis;

FIG. 5 is a flowchart of a process that may be implemented in one or more embodiments to train one or more models to generate a result that may be provided to a clinician to assist or inform the clinician's diagnosis;

FIG. 6 is a block diagram of a computing device with which one or more embodiments may operate.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Disclosed herein are techniques for evaluating the health of a subject (e.g., a patient), including techniques for generating one or more models (e.g., machine learning models) trained to predict (e.g., diagnose) one or more health conditions in a subject. Some such techniques include receiving audio data comprising speech of a subject and/or of one or more other persons, extracting vocal biomarkers of a subject from the audio data, and determining whether the vocal biomarkers correspond (or are likely to correspond, or are sufficiently likely to correspond, or otherwise satisfy a criteria for correspondence) to potential presence of one or more health conditions in the subject. Such extracting of vocal biomarkers and/or determining of presence of a health condition may be based at least in part on an analysis of the audio data of speech by one or more models, such as models generated or trained using machine learning. Some techniques also include receiving supplemental data (e.g., electronic health record (EHR) data) of one or more patients, extracting patient attributes from the EHR data, and determining whether the patient attributes correspond to a presence of a health condition based on an analysis by one or more models of audio data of speech of a patient together with the supplemental data. In some embodiments described herein, the vocal biomarker data analysis and the supplemental data analysis is performed by a multimodal model, though in other embodiments, vocal biomarker data analysis and supplemental data analysis is performed by separate models and the results of the separate models are combined.

Health conditions for which some embodiments may operate include neurological disorders (e.g., Parkinson's disease, Alzheimer's disease, Lou Gehrig's disease, and stroke rehabilitation), mental health (e.g., depression, anxiety, post-traumatic stress disorder, and bipolar disorder), cardiovascular and respiratory conditions (e.g., chronic obstructive pulmonary disease (COPD), asthma, and cardiac stress), developmental disorders (e.g., autism spectrum disorder and speech delays), metabolic and endocrine disorders (e.g., obesity, diabetes, and thyroid dysfunction), behavioral state (e.g., aggression, emotion), pain level, wellness (e.g., stress, mood), risk assessment (e.g., risk of imminent violent behavior), impairment (e.g., by alcohol, drugs, sleepiness, mental or physical fatigue), and/or any other health condition (transient, temporary, chronic, or otherwise) that can be reflected in the speech of a patient.

The inventors have recognized and appreciated that, conventionally, speech analysis has not been widely used for predicting health conditions and that a contributing factor to this has been the low resolution of the data available from traditional speech analytics. Higher data resolution often allows for more precise analysis and robust identification of subtle patterns associated with diseases or conditions, which leads to higher reliability. High resolution data has not, however, been traditionally available from speech.

Traditional speech data has had low information density because they rely on analysis only of the words included in the speech and discard other aspects of the speech (e.g., the audio of the speaking of those words). The average rate of speech for the average English speaker is approximately 150 words per minute, which in such conventional systems yields approximately 150 data points per minute.

This can result in what is often a mismatch between the available resolution of the data and analytical tools that are available for use in analyzing data so as to generate highly reliable results. For example, machine learning models, such as those based on some approaches to deep learning, are often designed to extract patterns from large and detailed datasets. When the input data is sparse or lacks the necessary resolution, these tools often underperform. For example, data of low resolution may result in overfitting or underfitting of a model, in which a model captures noise instead of meaningful patterns or fails to capture complexities altogether.

The inventors have further recognized and appreciated that speech could be advantageously used in machine learning driven diagnostics of health conditions if data resolution could be increased. The inventors determined that, rather than a word-based analysis, if audio data of speech were instead subjected to an acoustic analysis of speech, this may increase data resolution. For example, where conventional word-based analysis methods may produce 150 data points per minute, acoustic analysis of speech may produce several million data points per minute. This increase in resolution can provide data points usable by some machine learning models to provide reliable diagnostic analysis of speech. Vocal biomarkers might be derivable from audio data of speech using such acoustic analysis, where the vocal biomarkers may include objective measures of voice such as pitch, pitch variability, tremors (including microtremors) in speech, tone, rhythm, amplitude, speech rate, prosody, pause duration, respiratory markers, and/or other features characterizing the acoustics of the speech. The inventors have additionally recognized and appreciated that such vocal biomarkers may be used to diagnose health conditions or assist clinicians in diagnosis of health conditions, such as onset or progression of health conditions. Such diagnosis may be done using vocal biomarkers as a symptom or indicator of such health conditions. In fact, the inventors have recognized and appreciated that vocal biomarkers may in some cases be used to detect early-stage health conditions such as in the case of some neurological conditions like Alzheimer's Disease, Parkinson's Disease, and others. Speech analysis may therefore offer an earlier detection or improved reliability of early detection of certain diseases, above what is conventionally available to providers and patients for some such diseases. Earlier detection could provide for better management of a health condition and improved patient outcomes. As a result of the difficulties noted above, though, speech has not traditionally been used, preventing the realization of these benefits for patients and providers.

While the inventors have recognized and appreciated that acoustic analysis of speech and vocal biomarkers can be used to reliably detect health conditions in patients, the inventors have additionally recognized and appreciated that the reliability of vocal biomarker analysis for a patient could be further improved with analysis of supplemental health record data because health record data can provide a rich source of contextual and longitudinal information about the overall health status, risk factors, comorbidities, and the like, of the patient and/or groups of patients. Health record data may include EHR data, genomic data, demographic details, clinical history, and/or any other non-speech data relating to the health of one or more patients.

The inventors recognized and appreciated that, conventionally, due to the mismatch in data scale, the combination of features from speech data and supplemental health record data would have been impractical for joint analysis. For example, images may include many millions of pixels per image, and speech data, as used traditionally, would not have been alignable with such other health data, preventing or undermining analysis of the data together. As a result, speech data together with supplemental health data has also not been used, and this traditional siloed approach means the interplay of factors that influence health may not be fully analyzed in a medical context, potentially leading to missed early warning signs, misdiagnoses, and/or less effective interventions.

To address these limitations, the inventors determined that features can be extracted from speech data along with supplemental health record data, such as genomic data, EHR data, and/or other health information, to form a higher resolution, multimodal input that can be utilized by one or more machine learning models for training and inference. By integrating health record data with vocal biomarker analysis, the inventors realized that it is possible to cross-reference and correlate subtle vocal patterns with objective clinical indicators, genetic predispositions, and historical trends.

The inventors have therefore recognized and appreciated the utility of a multimodal approach to analyzing vocal biomarkers for identifying health conditions. Multimodal tools for analyzing vocal biomarkers can in some cases offer transformative benefits for clinical environments. Such tools may in some embodiments combine audio data of speech with other health information, such as genetic markers, EHR data, environmental influences, and/or physiological signals, which may create a comprehensive and/or context-aware diagnostic framework. This integration may enable more precise detection of diseases by cross-referencing biomarkers and identifying correlations that single-modality systems do not. In some clinical settings, multimodal systems could provide specific advantages. First, in some cases they can enhance diagnostic accuracy by reducing false positives and negatives, particularly for conditions that exhibit subtle or overlapping symptoms, such as distinguishing between Parkinson's and multiple sclerosis. Second, in some cases they can improve accessibility by deploying these tools in telehealth platforms, wearable devices, or smartphones, enabling real-time diagnostics even in remote or underserved regions. Third, the integration of multimodal data into automated systems can reduce dependency on specialists in some cases, allowing general practitioners to make informed decisions and prioritize referrals more effectively.

In some embodiments and beyond diagnostics, such tools may support continuous or periodic health monitoring. Ambient listening devices and integration of health record data may provide longitudinal insights into patient health, capturing dynamic changes and enabling timely interventions. Integration of health record data may be in real-time, contemporaneously, during a patient encounter, or within a relevant time window such as the same minute, ten-minute period, or thirty-minute period, and/or any other suitable integration with audio data. Moreover, multimodal systems may allow healthcare to be personalized by tailoring diagnostics and treatment plans based on a patient's unique genetic, environmental, and/or behavioral context. This capability may ultimately lead to better outcomes and more efficient use of healthcare resources.

Some techniques described herein may be useful in some embodiments in generating one or more models to output one or more health risk scores for one or more health conditions based on vocal biomarkers and enhanced by supplemental data from other, non-speech modalities. Further described herein are examples of techniques and systems with which such techniques may be used. These include, for example, (1) systems with which some embodiments of methods described herein may operate; (2) methods for conducting training of at least one multimodal model using information from two or more modalities (e.g., audio data of speech and EHR data) corresponding to a presence of a health condition; (3) methods of identifying one or more health conditions predicted by the at least one model; and (4) methods for training one or more models using supplemental data of the patient, such as genomic data and/or EHR data, to output a determination of whether one or more health conditions are present in a subject.

The following description and examples illustrate in detail some embodiments of techniques and technologies described herein. It is to be understood that embodiments are not limited to acting in accordance with the specific examples provided herein, as other approaches are possible. Those of skill in the art will recognize that there may be variations and modifications from the specific examples below that are within the scope of this disclosure.

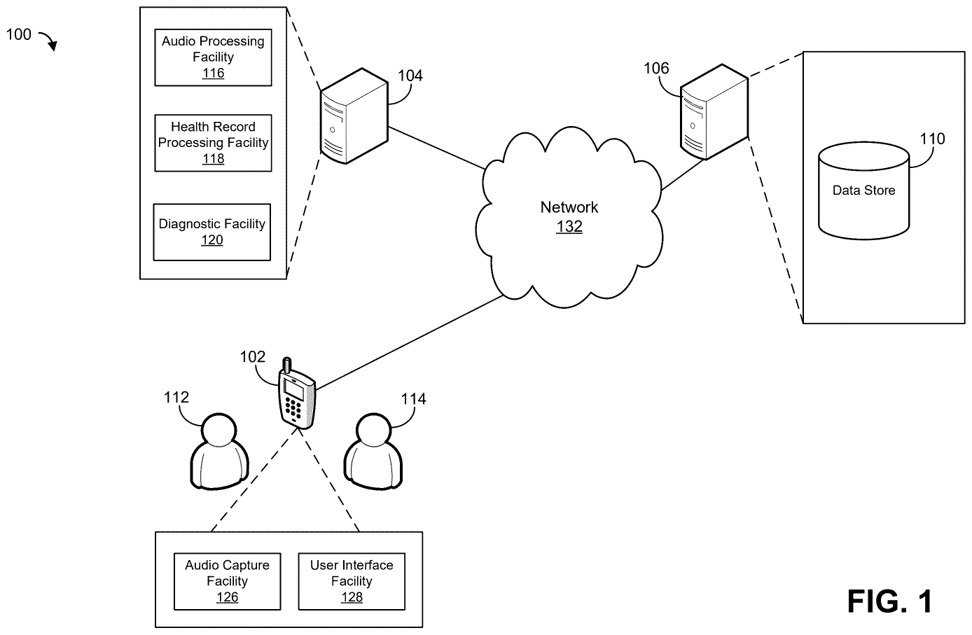

FIG. 1 is a block diagram of a system 100 with which one or more embodiments may operate. The system 100 may be used by a clinician 112 (e.g., a physician, nurse, researcher, technologist, technician etc.) and/or a subject 114 (e.g., a patient, clinical study participant, etc.) to diagnose the subject 114 with, or as part of a diagnostic evaluation of the subject 114 by a clinician for, one or more health conditions based on the subject's vocal biomarkers and supplemental health record data from, for example, health records of subject 114. In some embodiments, the health record data may additionally or alternatively be from health records of one or more other persons, or extracted or determined from health records of one or more other persons. Such information that may be extracted or determined from health records may include statistically-derived values or ranges (such as an average or a range covered by a standard deviation, or other value/range) for one or more health characteristics. For example, the health record data may be from health records of or regarding people in the same demographic group, geographic group, and/or medical/diagnostic group as the subject 114, or another group of which the subject 114 is a member or has members that share one or more characteristics with subject 114.

The subject 114 may be a human subject that can provide speech. Health conditions with which some embodiments described herein may operate may include, for example, neurological disorders (e.g., Parkinson's Disease, Alzheimer's Disease, mild cognitive impairment (MCI), amyotrophic lateral sclerosis (ALS), and multiple sclerosis (MS)), mental health and behavioral disorders (e.g., depression, anxiety disorders, bipolar disorder, schizophrenia and psychotic disorders), cardiovascular and respiratory diseases (e.g., chronic obstructive pulmonary disease (COPD), heart failure, hypertension, sleep apnea), developmental disorders (e.g., autism spectrum disorder, speech and language delays, Huntington's Disease), metabolic and endocrine disorders (e.g., diabetic neuropathy, thyroid disorders, obesity and metabolic syndrome), infections and autoimmune diseases (e.g., respiratory infections, Lupus, rheumatoid arthritis), and/or any other speech-manifesting health conditions. The system 100, in some embodiments, may be used to produce a degree of likelihood of the subject 114 having the one or more health conditions by analyzing a combination of audio data of speech and supplemental health information from, for example, health records with one or more trained machine learning (ML) models. The ML model(s) may have been trained on prior audio data and other health information such as health record data. Such prior audio data and other health information may be or include those from other subjects with one or more diagnosed health conditions.

The audio data that the system 100 can analyze may be audio data that includes speech or may be data that characterizes or relates to audio data of speech, such as that was derived through an acoustic analysis of speech.

The system 100 can include a client computing device 102, which may be a desktop or laptop computer, smart mobile phone, tablet, wearable (e.g., smart watch, device on a lanyard, smart glasses, or other wearable), server, or suitable device. The client computing device 102 may include an audio capture facility 126 and/or a user interface facility 128.

The audio capture facility 126 may connect with or operate one or more sensors for capturing audio, such as a microphone or microphone array, by which the facility 126 may receive audio data of speech. Such a microphone/array may be integrated with the client computing device 102 or separate from it and communicatively connected via wired and/or wireless communication. A microphone/array may, in some cases, be disposed in a room in which a conversation is taking place, such as an exam room or other room of a medical office in a case of a patient encounter. A microphone may be mounted on or integrated with a wall, ceiling, furniture, or other surface, or be worn by a clinician or subject. Embodiments are not limited to operating with a particular type of microphone.

The captured audio may be stored on the client computing device 102. Speech may be captured in any suitable manner. For example, the audio capture facility 126 may record speech provided by subject 114. In some cases, the speech may be speech spoken by the subject 114 in response to a prompt, such as a structured reading passage, a verbal fluency test, or a specific question designed to elicit speech for vocal biomarker analysis. In other cases, the speech may be speech spoken by the subject 114 during a discussion with clinician 112, such as a dialogue between the clinician 112 and subject 114 or during unstructured conversation, interviews, or other interactive scenarios. Speech also may be captured passively or ambiently, such as during daily activities or while the subject is engaged in conversation with others within range of the microphone. In some such cases, the captured audio data may be audio of two or more speakers, and in some cases filtering or speaker segmentation/diarization may be used to yield audio data for speech of the subject 114. Embodiments may include capturing speech specifically for the purpose of vocal biomarker analysis, as well as capturing incidental, spontaneous, or ambient speech, thereby supporting a wide range of speech collection modalities.

The user interface facility 128 enables the subject 114 or the clinician 112 to interact with the client computing device 102. The subject 114 or the clinician 112 can use the user interface facility 128 to provide data to the client computing device 102 such as medical data (which may be provided to the health record device 106), credentials (which may be used to access data from the health record device 106), initiate a diagnostic session with the diagnostic device 104, and/or any other input. The subject 114 or the clinician 112 can also use the user interface facility 128 to receive and/or display data by the client computing device 102 such as medical data (which may be received from the health record device 106), diagnostic results (which may be received from the diagnostic device 104) and/or any other output. The user interface facility 128 may be in any suitable format. In some examples it may be a web interface, such as one or more web pages into which values may be output and which may display results of a diagnostic analysis by the diagnostic device 104, but embodiments are not so limited. Other embodiments may use a mobile application, software application, or other software, firmware, or other computer instructions. The user interface facility 128 may accept input in a variety of different formats, such as through speech recognition, text input, or other means, as embodiments are not limited in this respect.

The system 100 may include a health record device 106, which may be a desktop or laptop computer, server, tablet, array/cluster of servers, or other suitable device or set of devices. The health record device 106 may include a data store 110.

The data store 110 may include one or more databases (e.g., relational, graph, time-series), file systems, object stores, key-value stores, warehouses, and/or any other health data repository that holds data in a structured and/or unstructured format. The data store 110 may be used by the health record device 106 to store electronic health records (EHR also sometimes called electronic medical records (EMR)), health data that has yet to be input into the EHR, remote health data (e.g., from a health tracker of a subject 114), and/or the like. The data may be structured (e.g., as electronic forms, spreadsheets, etc.) and/or unstructured (e.g., free text in handwritten notes, scanned documents, etc.). For example, the health records may include lab results, prescription lists, genetic information, family medical history, demographic information, and/or the like. The health records may also include medical literature, doctor's notes, billing codes, CPT codes, procedure codes, genetic sequencing information, call transcripts, prescription limits, test results, and/or the like. The health records may also include image files images (e.g., radiography, such as x-rays, CAT scans, MRI scans, or the like), video files, audio files, and/or the like. The health record device 106 may access the health records via an application programming interface (API), graphical user interface, file transfer protocol, remote desktop connection, web scraper, and/or the like.

The system 100 may include a diagnostic device 104, which may be a desktop or laptop personal computer, mobile device (e.g., smart mobile phone, tablet), server, or other suitable device or set of devices. The diagnostic device 104 may include an audio processing facility 116, a health record processing facility 118, and/or a diagnostic facility 120.

The audio processing facility 116 is configured to process audio (e.g., pre-captured or captured in real time, such as from an audio capture facility 126), identify speech data in the audio, identify different speakers, and/or process speech data to determine one or more vocal biomarkers. When audio is received, the audio processing facility 116 may perform speech detecting, discriminating between speech and non-speech data of the ambient audio data to filter out background noise, silence, or other irrelevant acoustic data. The audio processing facility 116 may also perform speaker diarization, segmenting the speech data of the ambient audio data by individual speaker. The audio processing facility 116 may also filter the speech data of one or more speakers to remove segments that are unlikely to contribute meaningful information for biomarker analysis. This may include brief utterances, filler words, or speech artifacts that lack sufficient prosodic, acoustic, or temporal complexity. This may additionally or alternatively include speech that does not include multiple different words, or where a meaning of words used in the speech does not satisfy at least one criterion (e.g., having a non-trivial meaning, such as expressing more than mere agreement (“yes” or “yes yes yes”) or mere disagreement (“no” or “no no no”)). Additionally or alternatively, audio that may not contribute meaningful information for biomarker analysis may include audio for which acoustic quality does not pass one or more criteria, such as having a signal-to-noise threshold below a threshold, being too quiet, or other criterion related to the acoustics of the audio. Generally speaking, a determination of whether segments of audio data are to be used for determining whether a subject has one or more health conditions may include evaluating whether segments of audio do (or do not) satisfy one or more conditions. The audio processing facility 116 may also subject the speech data of one or more speakers to feature extraction, where the speech data is transformed into speech data embeddings, which are multidimensional representations that encode certain vocal attributes. The embeddings may include representations of prosodic features (e.g., pitch, intonation, and rhythm), acoustic features (e.g., formant frequencies, spectral energy, and harmonics), temporal features (e.g., speech rate and pause duration), respiratory features (e.g., breath control or voice tremor), and/or other features indicative of vocal biomarkers.

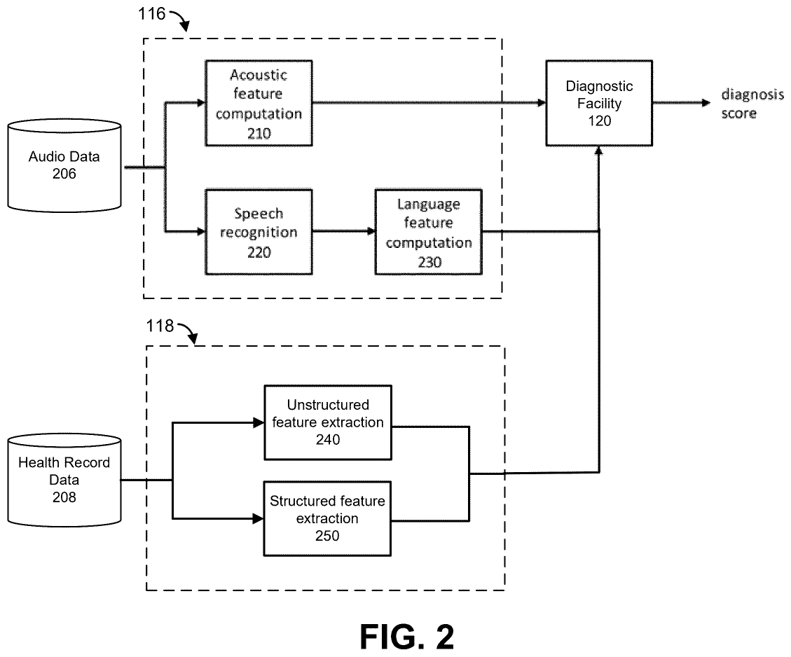

As discussed above, vocal biomarkers include measurable indicators from a subject 114 of some biological state and/or condition of the user. The biological state of the user may include the presence of a health condition and the condition of the user may include the user's quality of life. Vocal biomarkers may include objectively identifiable characteristics such as prosodic features, acoustic features, temporal features, and/or respiratory features in the speech data. Vocal biomarkers may be used to identify potential health conditions of the subject 114. Illustrative techniques for identifying vocal biomarkers are discussed in further detail below with respect to FIG. 2.

In some embodiments, one or more models (e.g., one or more deep learning models, or other techniques) may encode the speech data into structured embeddings for analysis with the diagnostic facility 120. In accordance with some techniques described herein, the audio data can be combined with other modalities for analysis to determine whether the subject 114 has one or more health conditions. In some such embodiments, these embeddings may capture latent vocal patterns, which may be linked to health conditions even in short speech samples and so may be usable to determine whether the subject 114 has any of the one or more health conditions. Illustrative techniques for identifying vocal biomarkers is discussed in further detail below with respect to FIG. 2.

The health record processing facility 118 is configured to obtain information from health records of one or more subjects. The information may be obtained from a health record device 106 via an application programming interface (API), graphical user interface, file transfer protocol, remote desktop connection, webpage scrape, and/or the like. The information may include files such as documents, spreadsheets, and/or the like, which may include patient records, textbooks, research papers, articles and other literature, doctor's notes, billing codes, CPT codes, procedures codes, genetic sequencing information, call transcripts, prescriptions lists, test results, lab results, and/or the like. The information may also include images (e.g., radiography such as x-rays, CAT scans, MRI scans, and/or the like), videos, audio, and/or the like.

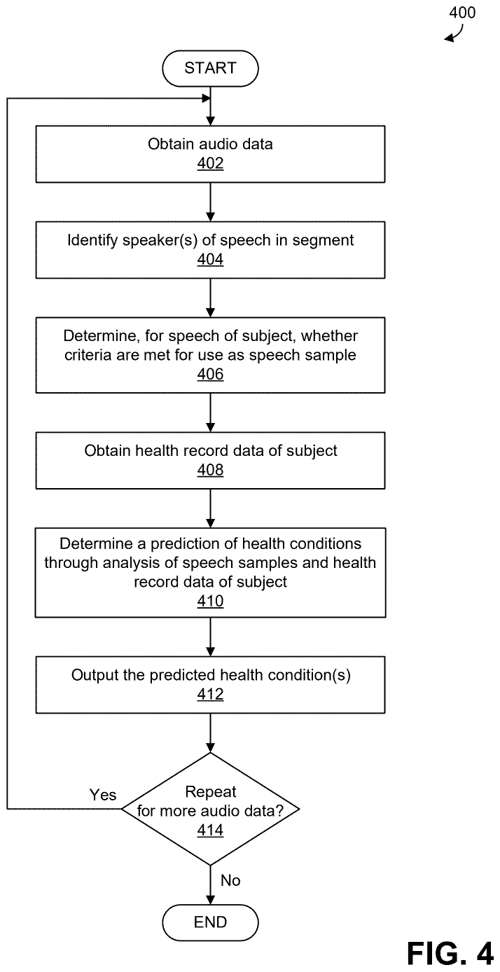

With the obtained information, the health record processing facility 118 may encode text, image, or other health data into structured embeddings, transforming health records into features that can be combined with audio data embeddings (e.g., from the audio processing facility 116). Features may include symptom descriptions (e.g., “patient reports tremors and slowed speech”), past medical history (e.g., prior strokes, cognitive decline), medication and side effects (e.g., drugs affecting speech patterns), lab test results and imaging reports, genetic markers or predispositions, and/or the like. Analyzing health records with one or more LLMs is described further below with respect FIG. 4. In some embodiments, the health record processing facility 118 may pre-process the health record data. Pre-processing may include data cleaning (e.g., removing or correcting errors), data normalization, feature selection, handling imbalanced data, text preprocessing (e.g., stemming, lemmatizing), handling missing values, and/or any other data pre-processing technique.

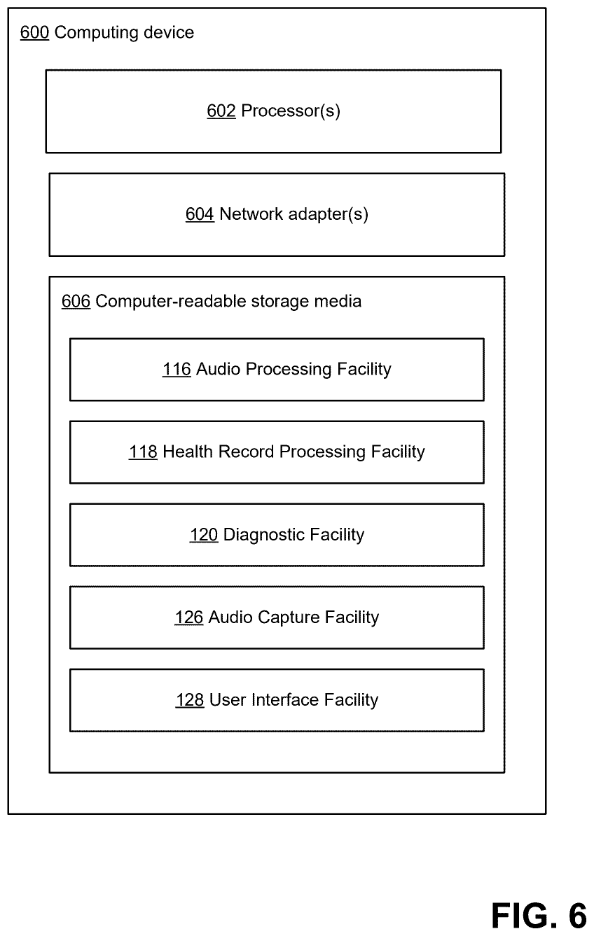

The diagnostic facility 120 is configured to generate a prediction of whether the subject 114 has one or more health conditions. To do so, the diagnostic facility 120 may receive the outputs (e.g., embeddings) of the audio processing facility 116 and the health record processing facility 118 and perform a combined analysis for predicting a diagnosis of the subject 114. Generating diagnoses based on the outputs of the audio processing facility 116 and/or the health record processing facility 118 is described in further detail below with respect to FIG. 6.

The diagnostic facility 120 may evaluate whether the vocal biomarkers (e.g., extracted from speech data) correspond to known vocal biomarkers (e.g., acoustic or prosodic features) of specific health conditions. These conditions may include, but are not limited to, anxiety, depression, Alzheimer's disease, Parkinson's disease, cardiovascular stress, fatigue, and other condition neurocognitive or emotional conditions. The diagnostic facility 120 may aggregate the vocal biomarker and health record results and cross-reference the results with known disease symptoms, indicators, or the like.

In some embodiments, the health record device 106 may provide medical literature, regulations, rules, manuals, policies, research papers, articles, journals, dissertations, speeches, lectures, and/or the like, to an LLM model that is trained to analyze scientific literature to determine a veracity of a health condition result (e.g., predicted by the audio processing facility 116) for a patient, including citations or references.

The diagnostic facility 120 may provide the outputs to a clinician 112, to a subject 114, or to another interested party (e.g., an insurance company). The diagnostic facility 120, for instance, may provide the results in an interface (e.g., a graphical user interface). For example, the diagnostic facility 120, during a conversation between a doctor and a patient, may present the analysis results on the doctor's device while the conversation is ongoing (e.g., in real-time). The results may indicate that a health condition for the patient has been detected, may provide a likelihood that the patient has the health condition, or the like, based on the patient's speech during the conversation.

In some embodiments, the results output from the diagnostic facility 120 may include one or more scores. The scores may include a score associated with the audio data analysis and another score associated with the health record analysis. The scores may reflect a confidence level or a degree of accuracy of the outcomes of the audio data analysis and the health record analysis. In some embodiments, the scores may include a score associated with an analysis of one duration of audio data and another score associated with another analysis of different duration of audio data. The scores may be combined into a single score, such as an average, a weighted average, a sum, or the like. The diagnostic facility 120 may provide the score(s) to a doctor, a patient, or the like.

The system 100 can include a network 132 to facilitate communications among the client computing device 102, the diagnostic device 104, and/or the health record device 106. The network 132 can be or include any one or more wired and/or wireless, local- and/or wide-area networks (which may be physical and/or virtual), including one or more enterprise networks and/or the Internet. The network 132 includes one or more servers, routers, switches, and/or other networking equipment.

While the example of FIG. 1 includes the client computing device 102, the diagnostic device 104, and the health record device 106 as separate devices, embodiments of the disclosure are not so limited and may include greater or fewer than the number of devices shown. In some embodiments, the system 100 may include one or more devices for each of the client computing device 102, the diagnostic device 104, and/or the health record device 106. For example, the diagnostic device 104 may be a cluster of devices on a cloud platform. In some embodiments, the operations performed by multiple facilities may be performed by a single facility, and vice versa. For example, the diagnostic facility 120 may perform the operations of the audio processing facility 116 and the health record processing facility 118. In some embodiments, the operations performed by multiple devices may be performed by a single device, and vice versa. For example, the client computing device 102 may store health records of the subject 114 and diagnose the subject 114 based on the stored health records.

FIG. 2 is a block diagram depicting an example system for processing speech data 206 and health record data 208 with a model (e.g., diagnostic facility 120) to predict a diagnosis, in accordance with one or more embodiments.

In processing the audio data, features may be computed from the speech data 206, and then the features may be processed by the model. Any appropriate type of features may be used.

The features may include acoustic features, where acoustic features are any features computed from the audio data that do not involve or depend on performing speech recognition on the audio data (e.g., the acoustic features do not use information about the words spoken in the speech data). For example, acoustic features may include mel-frequency cepstral coefficients, perceptual linear prediction features, jitter, or shimmer.

The features may include language features where language features are computed using the results of a speech recognition. For example, language features may include a speaking rate (e.g., the number of vowels or syllables per second), a number of pause fillers (e.g., “ums” and “ahs”), the difficulty of words (e.g., less common words), or the parts of speech of words following pause fillers.

The audio data 206 (e.g., from the audio capture facility 126) may be processed by acoustic feature computation facility 210 and/or speech recognition facility 220. Acoustic feature computation facility 210 may compute acoustic features from the audio data 206, such as any of the acoustic features described herein. Speech recognition facility 220 may perform automatic speech recognition on the audio data using any appropriate techniques (e.g., Gaussian mixture models, acoustic modelling, language modelling, and neural networks). In some embodiments, the speech recognition facility 220 may use pre-trained embedding models based on the audio signal.

Because speech recognition facility 220 may use acoustic features in performing speech recognition, some processing of these two components may overlap and thus other configurations are possible. For example, acoustic feature computation facility 210 may compute the acoustic features needed by speech recognition facility 220, and speech recognition facility 220 may thus not need to compute any acoustic features. In some embodiments, acoustic feature computation facility 210 may use various techniques for voice activity detection to detect that a person is speaking.

Language feature computation facility 230 may receive speech recognition results from speech recognition facility 220 and process the speech recognition results to determine language features, such as any of the language features described herein. The speech recognition results may be in any appropriate format and include any appropriate information. For example, the speech recognition results may include a word lattice that includes multiple possible sequences of words, information about pause fillers, and the timings of words, syllables, vowels, pause fillers, or any other unit of speech.

The features computed by acoustic feature computation facility 210 and/or language feature computation facility 230 may be the vocal biomarkers identified by the audio processing facility 116.

In processing the health record data 208, features may be computed from the health record data, and then the features may be processed by the model in addition to or instead of the features of the speech data. Any appropriate type of features may be used.

The diagnostic facility 120 may use other, non-speech features, in addition to acoustic features and language features from the audio data 206. For example, features may be obtained or computed from demographic information of a person (e.g., gender, age, or place of residence), information from a medical history (e.g., weight, recent blood pressure readings, or previous diagnoses), or any other appropriate information from health records associated with the subject 114.

The health record data 208 may be specific to a subject 114 and include one, some, or some combination of information regarding longitudinal health trends (e.g., disease progression, response to treatment), comorbidities and risk factors (e.g., preexisting conditions, family history), medication usage (e.g., drugs taken and their side effects), behavioral and lifestyle factors (e.g., substance use, occupational hazards, geographic location, socioeconomic status), genomic markers, text-based features (e.g., clinician notes, imaging reports, lab test results), and/or the like. The health record data 208 may also or instead be non-specific to the subject 114 and include one, some, or some combination of information regarding medical literature, research papers, clinical guidelines, and any other suitable medical information. In some embodiments, the health record data 208 may be specific to a subject 114 and/or one or more other subjects. For example, the health record data 208 may be specific to a cross section of the population. The cross section may be any suitable cross section based on any one characteristic or combination of characteristics, as embodiments are not limited in this respect. As one example, a group may be defined as Latino males in their 30s with a particular gene and a history of back pain.

The unstructured feature extraction facility 240 may be used to extract features from unstructured health records, such as free-text clinical notes, physician summaries, patient narratives, and any other non-standardized documentation. The unstructured feature extraction facility 240 may utilize natural language processing (NLP) techniques, such as those powered by LLMs. The unstructured feature extraction facility 240 may preprocessing the health records with operations such as tokenization, sentence segmentation, and stopword removal. Named entity recognition may be used to extract relevant medical terminology, including symptoms, diagnoses, treatments, and medications. The extracted features may then be embedded using vector representations such as transformer-based contextual embeddings (e.g., BERT) or static word embeddings (e.g., Word2Vec).

The structured feature extraction facility 250 may be used to extract features from structured health records, such as standardized, discrete data fields found in EHRs, laboratory results, genomics databases, and medical imaging metadata. These features may include numerical values (e.g., blood pressure, cholesterol levels, oxygen saturation), categorical data (e.g., disease codes, medication names, genetic markers), time-series measurements (e.g., heart rate variability over time), and/or the like. The structured feature extraction facility 250 may apply feature engineering techniques, such as standardization and categorical encoding, to prepare structured data for integration with the output of the unstructured feature extraction facility 240. For example, continuous variables (e.g., lab values, age) may be normalized to a standard scale, while categorical variables (e.g., diagnostic codes, medication names) can be converted into one-hot encodings or embedded using entity embeddings to capture relationships between categories. The structured feature extraction facility 250 may embed these numerical representations as input vectors that can be concatenated with other features, enabling compatibility with multimodal ML architectures.

The performance of diagnostic facility 120 may depend on the features computed by acoustic feature computation facility 210 and/or language feature computation facility 230. Further, a set of features that performs well for one health condition may not perform well for another health condition. For example, word difficulty may be a feature for diagnosing Alzheimer's disease but may not be useful for determining if a person has a concussion. For another example, features relating to the pronunciation of vowels, syllables, or words may be useful for Parkinson's disease but may be less useful for other health conditions. Accordingly, techniques are needed for determining a first set of features that performs well (e.g., meets one or more reliability criteria) for a first health condition, and this process may need to be repeated for determining a second set of features that performs well (e.g., meets one or more reliability criteria) for a second health condition.

The selection of features for diagnosing a health condition may be more important in situations where an amount of training data for training the machine learning model is relatively small. For example, for training a machine learning model for diagnosing concussions, the needed training data may include audio data of a number of individuals shortly after they experience a concussion. Such data may exist in small quantities and obtaining further examples of such data may take a significant period of time.

Training machine learning models with a smaller amount of training data may result in overfitting where the machine learning model is adapted to the specific training data but because of the small amount of training data, the model may not perform well on new data. For example, the model may be able to detect all of the concussions in the training data but may have a high error rate when processing production data of people who may have concussions.

One technique for preventing overfitting when training a machine learning model is to reduce the number of features used to train the machine learning model. The amount of training data needed to train a model without overfitting increases as the number of features increases. Accordingly, using a smaller number of features allows models to be built with a smaller amount of training data.

Where it is beneficial to train a model with a smaller number of features, it may be advantageous to select the features that will allow the model to perform well. For example, when a large amount of training data is available, hundreds of features may be used to train the model and it is more likely that appropriate features have been used. Conversely, where a small amount of training data is available, only 10 or so features may be used to train a model, and it is more important to select the features that are most important for diagnosing the health condition.

Described below are some examples of features that may be used to diagnose a health condition, in some embodiments. It should be appreciated that embodiments are not limited to operating with all of these features or with any particular combination of these features. Other embodiments may use other features.

Acoustic features may be computed using short-time segment features. When processing audio data, the duration of the audio data may vary. For example, some audio may be a second or two and other audio may be several minutes or more. For consistency in processing audio data, it may be processed in short-time segments (sometimes referred to as frames). For example, each short-time segment may be 25 milliseconds, and segments may advance in increments of 10 milliseconds so that there is a 15 millisecond overlap over two successive segments.

Short-time segment features may in some cases include one or more of the following examples: spectral features (such as mel-frequency cepstral coefficients or perceptual linear predictives); prosodic features (e.g., pitch, energy, or probability of voicing); voice quality features (e.g., jitter, jitter of jitter, shimmer, or harmonics-to-noise ratio); entropy (e.g., to capture how precisely an utterance is pronounced where entropy may be computed from the posteriors of an acoustic model that is trained on natural speech data).

The short-time segment features may be combined to compute acoustic features for the audio. For example, a two-second speech sample may produce 200 short-time segment features for pitch that may be combined to compute one or more acoustic features for pitch.

In some cases, short-time segment features may be combined to compute an acoustic feature for a speech sample. For example, in some implementations, an acoustic feature may be computed using statistics of the short-time segment features (e.g., arithmetic mean, standard deviation, skewness, kurtosis, first quartile, second quartile, third quartile, the second quartile minus the first quartile, the third quartile minus the first quartile, the third quartile minus the second quartile, 0.01 percentile, 0.99 percentile, the 0.99 percentile minus the 0.01 percentile, the percentage of short-time segments whose values are above a threshold (e.g., where the threshold is 75% of the range plus the minimum), the percentage of segments whose values are above a threshold (e.g., where the threshold is 90% of the range plus the minimum), the slope of a linear approximation of the values, the offset of a linear approximation of the values, the linear error computed as the difference of the linear approximation and the actual values, or the quadratic error computed as the difference of the linear approximation and the actual values). In some implementations, an acoustic feature may be computed as a speech embedding to represent the partial or full audio. The speech embedding may include identity vectors such as an i-vector or an x-vector of the short-time segment features and speech representation based on a self-supervised pre-trained model, such as wav2vec or Trillson. An identity vector may be computed using any appropriate techniques, such as performing a matrix-to-vector conversion using a factor analysis technique and a Gaussian mixture model for an i-vector or a neural network model for an x-vector.

Language features may in some cases include one or more of the following examples of features. A speaking rate, such as by computing the duration of all spoken words divided by the number of vowels or any other appropriate measure of speaking rate. A number of pause fillers that may indicate hesitation in speech, such as (1) a number of pause fillers divided by the duration of spoken words or (2) a number of pause fillers divided by the number of spoken words. A measure of word difficulty or the use of less common words. For example, word difficulty may be computed using statistics of 1-gram probabilities of the spoken words, such as by classifying words according to their frequency percentiles (e.g., 5%, 10%, 15%, 20%, 30%, or 40%). The parts of speech of words following pause fillers, such as (1) the counts of each part-of-speech class divided by the number of spoken words or (2) the counts of each part-of-speech class divided by the sum of all part-of-speech counts.

In some embodiments, language features may include a determination of whether a person answered a question correctly. For example, a person may be asked what the current year is or who the President of the United States is. The person's speech may be processed to determine what the person said in response to the question and to determine if the person answered the question correctly. Further, in some embodiments, language features may include a determination whether a person read correctly, e.g., read a presented passage correctly. In such an embodiment, the word error rate is computer and compared to the expected reading script, e.g., using an automatic speech recognition (ASR) result. In some embodiments, when the question prompt is intended to assess a verbal fluency test, e.g., asking the user to list the words in a category such as animals, an evaluation is performed to determine if the user's response actually belongs to the expected category by checking or calculating the distance between word vectors.

Health record features, or more generally, information regarding the health of the subject that is determined from health record data, may be used for refining predictions from acoustic and linguistic features. Such information may include, for example, demographic information, such as age, sex, education level, ethnicity, and socioeconomic status, which may influence speech patterns, cognitive function, and/or health risks. Health record features may also include genomic markers, such as single nucleotide polymorphisms (SNPs), polygenic risk scores, and epigenetic modifications, which can provide insights into predispositions for neurological, psychiatric, and/or metabolic disorders. Clinical history, such as diagnosed conditions, comorbidities, family history, and past medical events, may also be included. For example, a history of stroke or neurodegenerative disease could be correlated with specific speech impairments, reinforcing vocal biomarker findings. Medication and treatment records, such as the use of medications that can influence speech patterns. Cognitive and psychological assessments, such as scores from cognitive tests (e.g., Mini-Mental State Examination, MoCA) or psychiatric evaluations (e.g., PHQ-9 for depression), may also be included. Respiratory and cardiovascular data, such as pulmonary function tests, oxygen saturation levels, and cardiovascular markers, may also be included. Health record data may further include electronic health record notes, such as unstructured physician notes and structured clinical documentation, as well as functional and behavioral data, such as sleep patterns and physical activity levels.

To train a model for diagnosing a health condition, a corpus of training data (a “training corpus” or “training data”) may be collected. The training corpus may include examples of audio data and health record data where the diagnosis of the subject is known. For example, the rows of a table of may correspond to database entries. In this example, each entry includes an identifier of a person, the known diagnosis of the person (e.g., no concussion or a mild, medium, or severe concussion), and a filename of a file that contains the audio data and/or health record data. The training data may be stored in any appropriate format using any appropriate storage technology.

The training corpus may store a representation of audio and health record data of a subject using any appropriate format. For example, an audio data item of the training corpus may include digital samples of an audio signal received at a microphone (e.g., of an audio capture facility 126) or may include a processed version of the audio signal, such as mel-frequency cepstral coefficients.

A single training corpus may contain audio data and health record data relating to multiple health conditions, or a separate training corpus may be used for each health condition (e.g., a first training corpus for concussions and a second training corpus for Alzheimer's disease). A separate training corpus may be used for storing audio data and health record data for people with no known or diagnosed health condition, as this training corpus may be used for training models for multiple health conditions.

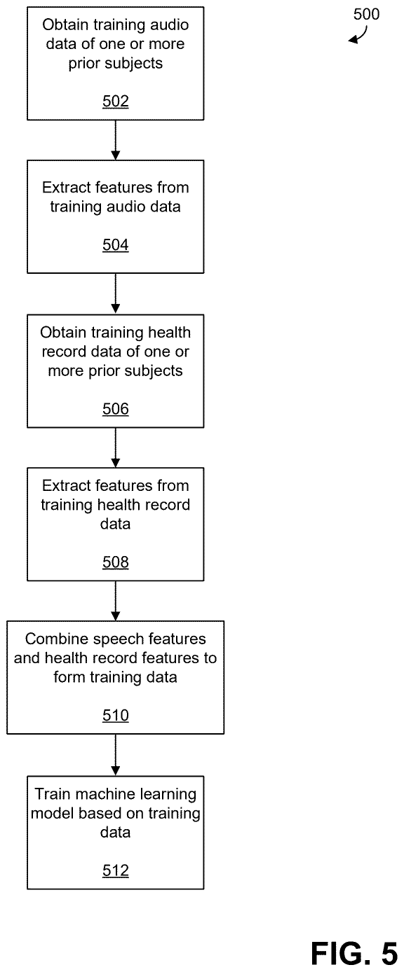

The diagnostic facility 120 may process the features (including acoustic features, language features, and/or health record features) with one or more machine learning models to output one or more diagnosis scores that indicate whether the subject 114 has a health condition described herein, such as a score indicating a probability that the subject 114 has the health condition and/or a score indicating a severity of the health condition. The diagnostic facility 120 may use any appropriate techniques, such as a multimodal classifier implemented with a support vector machine or a neural network, such as a multi-layer perceptron, a fully connected dense network, a convolutional neural network, and/or the like. Generating a diagnostic prediction is described in further detail below with respect to FIGS. 4 and 6. Some examples of training a machine learning model to generate a diagnostic prediction is described in further detail below with respect to FIG. 5.

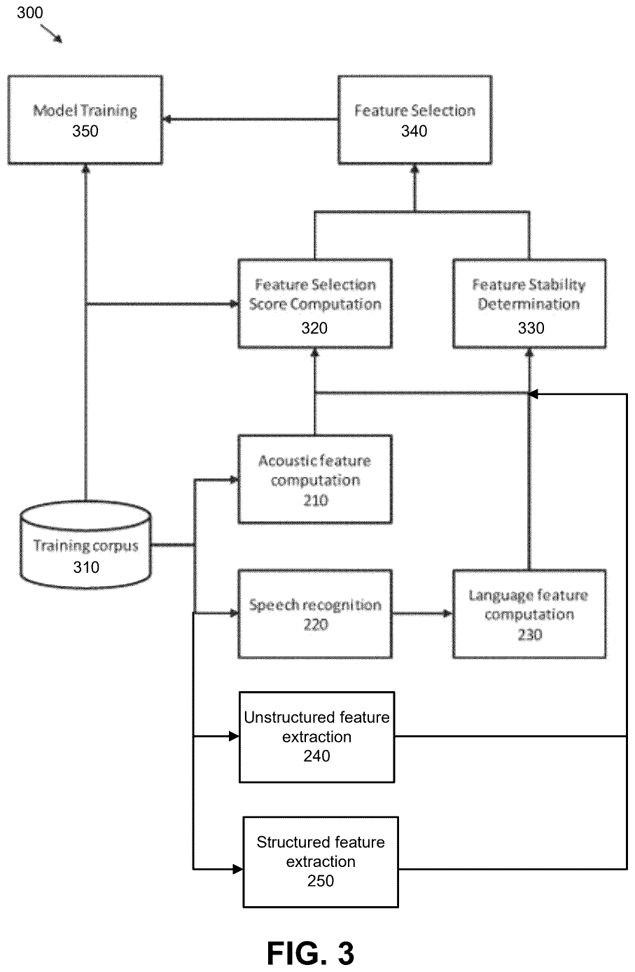

FIG. 3 depicts an example system 300 that may be used to select features for training a machine learning model of the diagnostic facility 120 for diagnosing a health condition based on audio data and/or health record data and using the selected features to train the machine learning model of the diagnostic facility 120. In some embodiments, system 300 may be used in different instances or different iterations to select features for different health conditions. For example, a first use of system 300 may select features for diagnosing concussions and a second use of system 300 may select features for diagnosing Alzheimer's disease (or other health conditions).

System 300 includes a training corpus 310 of audio data items for training a machine learning model for diagnosing a health condition. Training corpus 310 may include any appropriate information, such as audio data and/or health record data of multiple people with and without the health condition, a label indicating whether or not person has the health condition, and any other information described herein.

Unstructured feature extraction facility 240, structured feature extraction facility 250, acoustic feature computation facility 210, speech recognition facility 220, and/or language feature computation facility 230 may be implemented as described above to compute health record, acoustic, and language features for the health record and audio data in the training corpus. Unstructured feature extraction facility 240, structured feature extraction facility 250, acoustic feature computation facility 210, and language feature computation facility 230 may compute a large number of features so that the best performing features may be determined. This may be in contrast to the example of FIG. 2 where these components are used in a production system and thus these components may compute only the features that were previously selected.

Feature selection score computation component 320 may compute a selection score for each feature (which may be an acoustic feature, a language feature, or any other feature described herein). To compute a selection score for a feature, a pair of numbers may be created for each audio data item in the training corpus, where the first number of the pair is the value of the feature, and the second number of the pair is an indicator of the health condition diagnosis. The value for the indicator of the health condition diagnosis may have two values (e.g., 0 if the person does not have the health condition and 1 if the person has the health condition) or may have a larger number of values (e.g., a real number between 0 and 1 or multiple integers indicating a likelihood or severity of the health condition). Accordingly, for each feature, a pair of numbers may be obtained for each audio data item of the training corpus.

Feature selection score computation component 320 may compute a selection score for a feature using the pairs of feature values and diagnosis values. Feature selection score computation component 320 may compute any appropriate score that indicates a pattern or correlation between the feature values and the diagnosis values. For example, feature selection score computation component 320 may compute a Rand index, an adjusted Rand index, mutual information, adjusted mutual information, a Pearson correlation, an absolute Pearson correlation, a Spearman correlation, or an absolute Spearman correlation.

The selection score may indicate the usefulness of the feature in detecting a health condition. For example, a high selection score may indicate that a feature should be used in training the machine learning model, and a low selection score may indicate that the feature should not be used in training the machine learning model.

Feature stability determination component 330 may determine if a feature (which may be an acoustic feature, a language feature, or any other feature described herein) is stable or unstable. To make a stability determination, the audio data items may be divided into multiple groups, which may be referred to as folds. For example, the audio data items may be divided into five folds. In some implementations, the audio data items may be divided into folds such that each fold has an approximately equal number of audio data items for different genders and age groups.

The statistics of each fold may be compared to statistics of the other folds. For example, for a first fold, the median (or mean or any other statistic relating to the center or middle of a distribution) feature value (denoted as M₁) may be determined. Statistics may also be computed for the combination of the other folds. For example, for the combination of the other folds, the median of the feature values (denoted as Mₒ) and a statistic measuring of variability of the feature values (denoted as Vₒ), such as interquartile range, variance, or standard deviation, may be computed. The feature may be determined to be unstable if the median of the first fold differs too greatly from the median of the second fold. For example, the feature may be determined to be unstable if:

$${M}_{1} < {M}_{o} - C \frac{{V}_{o}}{2} or {M}_{1} > {M}_{o} + C \frac{{V}_{o}}{2}$$

where C is a scaling factor. The process may then be repeated for each of the other folds. For example, the median of a second fold may be compared with median and variability of the other folds as described above.

In some implementations, if, after comparing each fold to the other folds, the median of each fold within a predetermined threshold from the median of the other folds, then the feature may be determined to be stable. Conversely, if the median of any fold is outside the predetermined threshold from the median of the other folds, then the feature may be determined to be unstable.

In some implementations, feature stability determination component 330 may output a Boolean value for each feature to indicate whether the feature is stable or not. In some implementations, stability determination component 330 may output a stability score for each feature. For example, a stability score may be computed as the largest distance between the median of a fold and the other folds (e.g., a Mahalanobis distance).

Feature selection component 340 may receive the selection scores from feature selection score computation component 320 and the stability determinations from feature stability determination component 330 and select a subset of features to be used to train the machine learning model. Feature selection component 340 may select several features having the highest selection scores that are also sufficiently stable.

In some implementations, the number of features to be selected (or a maximum number of features to be selected) may be set ahead of time. For example, a number N may be determined based on the amount of training data, and N features may be selected. The selected features may be determined by removing unstable features (e.g., features determined to be unstable or features with a stability score below a threshold) and then selecting the N features with the highest selection scores.

In some implementations, the number of features to be selected may be based on the selection scores and stability determinations. For example, the selected features may be determined by removing unstable features, and then selecting all features with a selection score above a threshold.

In some implementations, the selection scores and stability scores may be combined when selecting features. For example, for each feature a combined score may be computed (such as by adding or multiplying or otherwise arithmetically combining the selection score and the stability score for the feature) and features may be selected using the combined score.

Model training component 350 may then train a machine learning model using the selected features. For example, model training component 350 may iterate over the health record and audio data items of the training corpus, obtain the selected features for the health record and audio data items, and then train the machine learning model using the selected features. In some implementations, dimension reduction techniques, such as principal components analysis or linear discriminant analysis, may be applied to the selected features as part of the model training. Any appropriate machine learning model may be trained, such as any of the machine learning models described herein.

In some implementations, other techniques, such as wrapper methods, may be used for feature selection or may be used in combination with the feature selection techniques presented above. Wrapper methods may select a set of features, train a machine learning model using the selected set of features, and then evaluate the performance of the set of features using the trained model. Where the number of possible features is relatively small and/or training time is relatively short, all possible sets of features may be evaluated, and the best performing set may be selected. Where the number of possible features is relatively large and/or the training time is a significant factor, optimization techniques may be used to iteratively find a set of features that performs well. In some implementations, a set of features may be selected using system 300, and then a subset of these features may be selected using wrapper methods as the final set of features.

FIG. 4 is a flowchart of a process 400 that may be implemented in one or more embodiments to evaluate audio data and/or health records for identifying features related to a health condition and generating a diagnostic result. For explanatory purposes, the figure is described with reference to the system 100 of FIG. 1 and thus the process 400 may be a computer-implemented method. However, this is merely illustrative, and features of the system 100 may be performed by any other system for implementing the subject technology. The operations of the process 400 need not be performed in the order shown, and one or more operations of the process 400 need not be performed or can be replaced by other operations.

At operation 402, the audio processing facility 116 obtains audio data. Obtaining audio data may involve obtaining audio recordings from various contexts where speech data is generated, such as clinical encounters between doctors and patients, phone calls between call center agents and callers, or other conversational settings. The audio data may be sourced from pre-recorded audio files (e.g., uploaded via the user interface facility 128), captured in real-time during interactions (e.g., by the audio capture facility 126), or generated in response to a prompt designed to elicit speech for vocal biomarker analysis.

In the context of clinical encounters, the audio data may be collected during consultations between care providers and patients. For example, a patient discussing symptoms with a physician or answering diagnostic questions may generate speech data that reflects vocal biomarkers associated with specific health conditions. An audio capture facility 126 may capture the conversational audio using microphones integrated into clinical equipment, wearable devices, or ambient listening systems installed in the consultation room.

Similarly, in the context of phone calls between call center agents and callers, an audio capture facility 126 may obtain audio data from recorded customer service interactions. For instance, a caller calling a support line to report an issue or seek assistance may exhibit vocal characteristics indicative of health conditions. The diagnostic device 104 may access the recordings through call center systems that store audio files for quality assurance or training purposes. The audio capture facility 126 may also or instead capture real-time audio during ongoing calls.

The diagnostic device 104 may also obtain audio data from other conversational settings, such as interviews or group discussions, where the subject was speaking to another person within range of a microphone. Additionally, ambient monitoring systems, such as smart speakers or wearable devices, may continuously record speech data throughout the day, capturing natural interactions that reflect the subject's vocal characteristics in various contexts.

In some embodiments, the audio processing facility 116 may preprocess the audio data to enhance the quality of the audio data. Preprocessing may include normalization to adjust the amplitude of the audio signal, noise cancellation to remove background interference, and/or vocal amplification to enhance the audibility of the speaker's voice. For example, in a clinical encounter, the audio capture facility 126 may filter out certain ambient sounds such as the hum of medical equipment or conversations from nearby rooms. Similarly, in a phone call scenario, the audio capture facility 126 may suppress static or line noise to focus on the speaker's voice. Additionally, non-speech portions of the audio, such as silence or irrelevant sounds (e.g., coughing or chair creaking), may be removed so that the samples are primarily speech.

Once the audio data is obtained, the audio processing facility 116 may obtain (e.g., extract) segments of the audio data that include a speech sample. The audio processing facility 116 may divide the audio data into discrete segments of audio data including a speech sample, where each speech sample corresponds to one or more utterances. The audio processing facility 116 may utilize techniques such as voice activity detection (VAD) to identify the start and end points of each utterance so that the speech samples are accurately segmented.

At operation 404, the audio processing facility 116 analyzes each segment of audio data to identify one or more speakers in each segment. The audio processing facility 116 may identify one or more speakers in each segment through diarization.

An approach to diarization may involve role recognition, which assigns roles to speakers based on the context and/or content of the conversation. For example, in a clinical encounter, the audio processing facility 116 may transcribe the audio data into text and use NLP to analyze the text for linguistic patterns and contextual cues. The audio processing facility 116 can then assign roles such as “doctor” and “patient” based on the distinct ways these types of individuals typically communicate. For instance, a doctor's speech may include medical terminology and diagnostic questions, while a patient's speech may consist of symptom descriptions and personal health concerns. Another approach to diarization may involve utilizing input channels. This approach may be used in phone call scenarios, where the audio data is captured separately for each participant. For instance, the audio processing facility 116 may attribute the caller's input to the caller and the agent's input to the call center agent. By using the distinct audio streams from each input channel, the audio processing facility 116 can accurately segment the audio data without requiring additional processing to distinguish between speakers.

Another approach to diarization may involve voice prints or vocal signatures. Voice prints may be or include unique acoustic characteristics associated with an individual's speech, such as pitch, tone, and cadence. The audio processing facility 116 may analyze acoustic characteristics to identify and differentiate speakers in the audio data. For example, if a clinical encounter involves a doctor and a patient, the device may use pre-recorded voice samples and/or real-time voice analysis to match a speaker's voice print to their respective role.

Once the speech data samples are assigned to a particular speaker, the diagnostic device 104 can focus the remainder of the analysis on the segments of audio data associated with the relevant speaker. For example, in a clinical encounter, the diagnostic device 104 may prioritize the patient's speech data for vocal biomarker analysis while disregarding the doctor's speech. Similarly, in a call center interaction, the diagnostic device 104 may analyze the caller's speech data to assess while disregarding the agent's speech.

At operation 406, the audio processing facility 116 evaluates each segment to determine whether at least some audio data of the speech of the subject included within the segment satisfies one or more criteria for use as a speech sample in vocal biomarker analysis.

The audio processing facility 116 may evaluate segments to determine whether the segments are sufficiently long. In some embodiments, the audio processing facility 116 may apply criteria such as minimum duration thresholds (e.g., three seconds) so that the samples are long enough to capture meaningful vocal patterns.

The audio processing facility 116 may evaluate segments to determine whether they include sufficiently meaningful speech samples. In some embodiments, segments of audio data with one- or two-word utterances may be discarded because those segments lack sufficient acoustic or prosodic complexity for meaningful analysis. For example, a brief response like “yes” or “no” may not provide enough information about pitch, tone, or rhythm to extract reliable biomarkers. In some embodiments, segments of audio data that include utterances that lack meaning (e.g., gibberish or non-sensical phrases) may be discarded.

The audio processing facility 116 may evaluate segments to determine whether their speech samples have excessive noise. In some embodiments, excessive noise includes overlapping speech and/or background noise. For instance, in a group discussion setting, if multiple participants speak simultaneously, the audio processing facility 116 may discard the segments of audio with overlapping speech samples to focus on those with clear, isolated utterances.

The audio processing facility 116 may evaluate segments to determine whether they have adequate audio quality. In some embodiments, inadequate audio quality includes segments of audio data that include distorted or muffled speech or high noise. For example, if a patient's voice is obscured by a malfunctioning microphone during a clinical encounter, the audio processing facility 116 may discard that segment to maintain the integrity of the analysis.

It should be understood that segment length, the meaning of speech samples, noise, and audio quality are merely examples and that other quality criteria are contemplated.

After evaluating the segments of speech for the relevant criteria, the audio processing facility 116 may combine the samples (if more than one) that satisfy the criteria as part of the aggregated audio data of speech of the subject.

In some embodiments, the audio processing facility 116 may add a buffer between samples before combining them. The buffer may help ease any abrupt changes in tone, volume, and/or cadence that could otherwise disrupt the continuity of the input speech data. For example, if one sample ends with a loud, emphatic statement and the next sample begins with a soft, hesitant response, the buffer can help normalize the transition to create a more cohesive audio segment. The buffer may include a brief pause (e.g., 50 ms) or a gradual adjustment in volume levels.

This operation may involve continuously reviewing incoming or available segments and, for each segment that meets the established standards (e.g., sufficient duration, sufficient audio quality, and absence of overlapping speech or excessive background noise) adding the sample to the set of one or more samples intended for vocal biomarker analysis.

For example, if a segment of audio data includes the subject speaking clearly for five seconds without interruption or significant background noise, and the utterance is more than a simple affirmation or negation, the audio processing facility 116 may include this segment in the set. Similarly, if another segment includes a longer response with diverse word usage and low noise, it may also be selected to be added to the set and/or aggregated with other segments.

In some embodiments, the audio processing facility 116 may continue to add speech data samples to the aggregated speech data (e.g., operations 404-406) until the aggregated speech data satisfies a predetermined threshold duration so that the input speech data is sufficiently informative for vocal biomarker analysis.

The threshold length may be determined based on the requirements of the machine learning model and/or the nature of the analysis. For instance, the audio processing facility 116 may combine samples to form input data points of approximately 30 to 40 seconds in duration, as this length may be sufficient to capture meaningful vocal biomarkers such as pitch variability, prosody, and pause duration. If the aggregated segments are shorter than the threshold length, the audio processing facility 116 may continue to add additional segments until the threshold length is satisfied.

At operation 408, the health record processing facility 118 obtains health record data. The health record processing facility 118 may interface with one or more health record devices 106, which may include EHR systems, hospital information systems, laboratory information management systems, genomic databases, and/or any other clinical data source. The health record processing facility 118 may access the health record devices 106 through standardized application programming interfaces (APIs), such as those conforming to HL7 FHIR (Fast Healthcare Interoperability Resources) standards, or through proprietary APIs provided by the healthcare institution. In some cases, the health record processing facility 118 may utilize secure file transfer protocols (SFTP), direct database queries, or web scraping techniques to extract relevant data when APIs are unavailable or insufficient.

The types of health record data obtained may be broadly categorized into structured and unstructured data. Structured data includes, e.g., discrete, codified information such as demographic details (e.g., age, sex, ethnicity), diagnosis codes (e.g., ICD-10), procedure codes (e.g., CPT), laboratory test results (e.g., LOINC-coded values), medication lists, vital signs, and time-series measurements. The health record processing facility 118 may query specific database tables or fields to retrieve this information, and may filter by subject identifiers, date ranges, or clinical encounter types to improve relevance to the training task.

Unstructured data includes, e.g., free-text clinical notes, physician summaries, discharge reports, imaging narratives, and any other narrative documentation. To obtain this data, the health record processing facility 118 may extract text fields from EHR systems or document management systems. In some implementations, the health record processing facility 118 may perform optical character recognition (OCR) to digitize handwritten or scanned documents. The health record processing facility 118 may also retrieve associated metadata including, e.g., document timestamps, author information, and document type, to provide additional context for downstream processing.

In addition to or instead of EHR data, the health record processing facility 118 may obtain other health record data, which includes, e.g., genomic data (e.g., single nucleotide polymorphisms, polygenic risk scores), imaging data (e.g., DICOM files from radiology systems), and/or data from wearable devices or remote monitoring systems. For genomic data, the health record processing facility 118 may interface with laboratory information systems or external genomic databases, retrieving information such as variant call files (VCFs), genetic test reports, or structured genetic risk scores. For imaging data, the facility may access picture archiving and communication systems (PACS) and retrieve for example image files and radiology reports.

The health record processing facility 118 may handle data from multiple institutions or sources, which may use different data schemas, coding systems, or storage formats. To address this, the health record processing facility 118 may perform data normalization and/or harmonization that, e.g., map disparate coding systems to a common ontology (e.g., mapping local diagnosis codes to ICD-10, or medication names to RxNorm). The health record processing facility 118 may also or instead perform data cleaning steps to, e.g., correct errors, handle missing values, and/or standardize units of measurement.

In some embodiments, the health record processing facility 118 may perform batch data extraction, where the facility periodically retrieves large datasets for offline model training, and/or real-time streaming, where new health record data is ingested as it is created.

As discussed above, in some embodiments the health record data may be health record data of the subject. In other embodiments, the health record data may additionally or alternatively include health record data regarding one or more other persons, such as a group of which the subject is a member. Such a group may be a demographic group of people (e.g., people sharing one or more demographic characteristics), a geographic group (e.g., people sharing one or more geographic characteristics, such as currently or previously living in a geographic area), medical or diagnostic characteristics (e.g., people sharing one or more diagnoses or medical conditions, or having a medical characteristic such as one or more genes or polymorphisms, one or more disabilities or medical limitations, or one or more other medically-relevant variations from reference anatomy or reference health), or any other group of people that share one or more characteristics. In a case that health record data includes data of one or more persons, the health record data may include values or ranges of values for one or more characteristics. In some such cases, the health record data may have been extracted from or derived from health records, but may itself be stored in one or more other databases and not in a health record for a specific individual, patient, or subject.

At operation 410, the diagnostic device 104 determines a prediction of whether a subject has any of one or more health conditions by analyzing speech data from operation 402-406 and health record data from operation 408.

The audio processing facility 116 may extract vocal biomarkers from the subject's speech data. The vocal biomarkers may include prosodic features (e.g., pitch, tone, and rhythm), acoustic features (e.g., amplitude and frequency), temporal features (e.g., speech rate and pause duration), and respiratory features (e.g., breath control or voice tremor). The extracted features may be encoded into embeddings that capture the relevant vocal biomarkers. Generating embeddings may be performed with models such as XVector or HuBERT.

The health record processing facility 118 may extract features from health record data, which may include structured data (e.g., demographic details, diagnosis codes, lab results, medication lists, and time-series measurements) and unstructured data (e.g., physician notes, discharge summaries, and imaging narratives). In some embodiments that use health record data for other people (e.g., a group of which the subject is a member), the health record data may be provided to the health record processing facility 118 as health record data for the group and identified as group data and that the subject is a member of the group. This may be separate from health record data specifically of or for the subject. In other embodiments, thought, the health record data for a group may be identified as health record data for the subject, given that the subject is a member of the group. For example, one or more values or ranges from the group health record data may be identified as values for health characteristics of the subject. This may, in some cases, be done in response to a determination that health record data for the subject does not include a particular value or characteristic, such that the value or range from the group health record data may be used in place of a missing value for the subject. In some such cases, a particular value may be selected from the group health record data, such as an average or median value from a range indicated by the health record data, a randomly-selected value from a range indicated by the health record data, or other manner of selecting a value from a range.

The health record features may also be encoded into structured embeddings, which may be generated using NLP techniques, including LLMs for unstructured text. In some embodiments, health record embeddings may be generated using LLMs in combination with focusing prompts to extract clinically relevant information from structured and/or unstructured health record data. To guide the LLM's analysis, a focusing prompt may be constructed. The focusing prompt may be designed to elicit clinically relevant information from the health record data, for example: “Given this medical record, what is the likelihood this patient has [target condition]?” or “Does this patient exhibit clinical features consistent with [disease]?” The prompt may also include instructions (e.g., few-shot examples) for the desired output format, such as a probability score, categorical label, or natural language explanation.

In some embodiments, retrieval augmented generation (RAG) may be employed. In RAG, the LLM is provided with additional context retrieved from health record data, external knowledge bases, population-level data, and/or relevant medical literature. This retrieval step enables the LLM to have access to up-to-date clinical guidelines, research findings, or cohort trends that may inform its analysis of the health record data.

The health record data, together with a focusing prompt and/or any retrieved context (if applicable), may then be input to the LLM. The LLM processes the information and generates a response, which may include a diagnostic assessment, risk score, or summary of relevant clinical features. The output from the LLM may then be encoded as an embedding, for example, by using the response text directly as a feature, by passing the response through an embedding model, or by extracting the penultimate neural layer activations from the LLM as a dense vector representation.

The diagnostic device 104 may provide the extracted vocal biomarkers and health record features to the diagnostic facility 120 where they may be combined and/or analyzed using one or more trained machine learning models. Once the embeddings are generated, the diagnostic device 104 may align and/or combine the vocal biomarker embeddings and health record feature embeddings. This may be accomplished by concatenating the embeddings into a unified feature vector or by using multimodal fusion techniques, such as attention-based neural network layers or transformer-based architectures. In some embodiments, separate scores may be generated for each modality (e.g., a vocal biomarker score and a health record score), which may then be combined to produce a composite assessment.

The combined feature vectors may then be input to one or more trained machine learning models within the diagnostic facility 120. These models, which may include neural networks, support vector machines, or any other supervised learning architectures, may be developed using training data that includes prior audio data and prior health record data from prior subjects (e.g., patients), along with labels indicating whether each subject had one or more health conditions, which is described in further detail below with respect to FIG. 5. The training process may involve selecting features that are most predictive of specific health conditions so that the models are optimized for accuracy and reliability, as described above with respect to FIG. 3. The one or more machine learning models evaluate the vocal biomarkers and/or the health record data to identify patterns or correlations indicative of health conditions, such as neurological disorders, mental health issues, cardiovascular stress, or respiratory impairments.

At operation 412, the diagnostic device 104 outputs the prediction of whether the subject has any of the one or more health conditions. Outputting the prediction may include presenting the results in a format accessible to relevant stakeholders, such as clinicians, subjects, and/or other authorized parties. The output may include a detailed analysis of the subject's speech data, highlighting the likelihood and/or severity of specific health conditions based on the extracted vocal biomarkers and/or the extracted health record features. The diagnostic device 104 may provide the predictions to the client computing device 102 to be displayed via a user interface facility 128, such as a clinician's desktop, tablet, or smartphone, enabling real-time access to diagnostic insights during a clinical encounter. In some embodiments, the diagnostic device 104 may also provide an indication of an activity in which the subject was engaged when the analyzed samples were captured.

The prediction may include one or more scores that quantify the confidence level and/or severity of the detected health conditions. For example, the diagnostic device 104 may provide a probability score indicating the likelihood that the subject has a particular condition and/or a severity score reflecting the extent of the condition. These scores may be derived from the analysis of the vocal biomarkers and may be presented alongside visual aids, such as charts, graphs, or tables, to facilitate interpretation. For example, a chart may display a probability score in a time series. The prediction may also include one or more citations to particular health record data supporting the prediction.

In some embodiments, the diagnostic device 104 integrates the prediction into the subject's EHR. This integration may include uploading the analyzed speech data, a transcription of the audio, the supporting health record data, the diagnostic results, and/or metadata about the machine learning models used in the analysis.

In some embodiments, the diagnostic device 104 may provide the prediction in real-time during ongoing interactions, such as a conversation between a clinician and a subject. This allows clinicians to receive live feedback on potential health conditions without interrupting the natural flow of the encounter. In some embodiments, the prediction may be delivered through automated systems, such as chatbots or call center platforms, where the results can be used to inform next steps, such as recommending further clinical evaluation.

At operation 414, the operations 402-412 may be performed iteratively over time. As new audio data becomes available, the audio processing facility 116 may analyze its segments, identify relevant speakers, evaluate the quality and/or content of the speech, and aggregate additional samples as appropriate and the diagnostic facility 120 may determine and output a prediction of health conditions based on the aggregated samples and/or health record data.

This iterative approach may be implemented using a sliding window of time. That is, the prediction may be determined using a first set of segments of audio data and a first set of health record data (e.g., time series data) in one iteration of the process, and the prediction may be determined using a second set of segments of audio data and second set of health record data in the next iteration of the process, where a set includes one or more segments or portions thereof. In some embodiments, the first and second set of segments may overlap. In some embodiments, the second set of segments of audio data and/or health record data may be a completely different set obtained after the first set of segments of audio data and/or health record data.

For example, as each new segment is processed, the audio processing facility 116 may update the aggregated set of speech samples by including the most recent qualifying segments and, if desired, removing the oldest segments to maintain a consistent window duration. In this way, the analysis remains current and responsive to changes in the subject's speech patterns over time. The health record processing facility 118 may similarly update the health record data to include the most recent data (e.g., in the case of real-time health record data) and remove data outside the window. This way, the audio data and the health record data can be analyzed for the same window of time.

The process 400 may repeat in this iterative manner until the monitoring session is terminated, providing ongoing, up-to-date insights into the subject's health status. In some embodiments, the process 400 may repeat in this iterative manner according to a predetermined schedule (e.g., once every four hours). Running the process 400 according to a predetermined schedule may also control when microphones are capturing audio data.

FIG. 5 is a flowchart of a process 500 that may be implemented in one or more embodiments to train one or more models to generate a diagnostic result. For explanatory purposes, the figure is described with reference to the system 100 of FIG. 1 and thus the process 500 may be a computer-implemented method. However, this is merely illustrative, and features of the system 100 may be performed by any other system for implementing the subject technology. The operations of the process 500 need not be performed in the order shown, and one or more operations of the process 500 need not be performed or can be replaced by other operations. For purposes of this description, the diagnostic device 104 trains the one or more models to generate a diagnostic result; however, it is contemplated that other devices may train the one or more models and the trained one or more models may be provided to the diagnostic device 104 for inference.

At operation 502, the diagnostic device 104 obtains prior audio data of one or more prior subjects. Obtaining prior audio data may involve obtaining audio recordings from various contexts where speech data is generated, such as clinical encounters between doctors and patients, phone calls between call center agents and callers, or other conversational settings. The prior audio data may be sourced from pre-recorded audio files or captured in real-time during interactions.

In the context of clinical encounters, the prior audio data may be collected during consultations between care providers and patients. For example, a patient discussing symptoms with a physician or answering diagnostic questions may generate speech data that reflects vocal biomarkers associated with specific health conditions. An audio capture facility 126 may capture the conversational audio using microphones integrated into clinical equipment, wearable devices, or ambient listening systems installed in the consultation room.

Similarly, in the context of phone calls between call center agents and callers, an audio capture facility 126 may obtain audio data from recorded customer service interactions. For instance, a caller calling a support line to report an issue or seek assistance may exhibit vocal characteristics indicative of health conditions. The diagnostic device 104 may access the recordings through call center systems that store audio files for quality assurance or training purposes. The audio capture facility 126 may also or instead capture real-time audio during ongoing calls.

The diagnostic device 104 may also obtain prior audio data from other conversational settings, such as interviews, group discussions, and/or ambient monitoring systems. For example, audio captured during a focus group discussion may provide insights into the vocal biomarkers of participants with known health conditions. Ambient monitoring systems, such as smart speakers or wearable devices, may continuously record audio data throughout the day, capturing natural interactions that reflect the subject's vocal characteristics in various contexts.

At operation 504, the audio processing facility 116 extracts features from the prior audio data. If the prior audio data is not specific to a particular subject, the audio processing facility 116 may first extract speech data samples specific to the particular subject from the prior audio data.

Extracting samples may include dividing the prior audio data into discrete speech data samples, where each speech data sample corresponds to one or more utterances. An utterance may be a continuous segment of speech spoken by a single speaker without interruption. For example, in a clinical encounter, an utterance might be a patient describing their symptoms in a single sentence, while in a call center interaction, an utterance could be a customer asking a question or providing feedback. The audio processing facility 116 may utilize techniques such as VAD to identify the start and end points of each utterance so that the samples are accurately segmented.

In some embodiments, after extracting the speech data samples from the prior audio data, the audio processing facility 116 may filter the speech data samples to remove those that are not conducive to (e.g., reduce the quality of) vocal biomarker analysis. Samples that are too short, such as one- or two-word utterances, may be discarded because they lack sufficient acoustic or prosodic complexity for meaningful analysis. For example, a brief response like “yes” or “no” may not provide enough information about pitch, tone, or rhythm to extract reliable biomarkers.

Similarly, samples with excessive noise or overlapping speech may be excluded to prevent interference with the analysis. For instance, in a group discussion setting, if multiple participants speak simultaneously, the audio processing facility 116 may discard the overlapping segments and focus on clear, isolated utterances. Samples with poor audio quality, such as those with distorted or muffled speech or high signal to noise ratio, may be removed from the dataset. For example, if a patient's voice is obscured by a malfunctioning microphone during a clinical encounter, the audio processing facility 116 may exclude that segment to maintain the integrity of the analysis. Additionally, the audio processing facility 116 may apply criteria such as minimum duration thresholds (e.g., three seconds) so that the samples are long enough to capture meaningful vocal patterns.

In some embodiments in which audio data includes multiple speakers, the audio processing facility 116 segments prior speech data samples (or prior audio data) by speaker so that the analysis focuses on the relevant subject's speech. This segregation may be achieved through diarization.

An approach to diarization may involve role recognition, which assigns roles to speakers based on the context and/or content of the conversation. For example, in a clinical encounter, the audio processing facility 116 may transcribe the prior audio data into text and use NLP to analyze the text for linguistic patterns and contextual cues. The audio processing facility 116 can then assign roles such as “doctor” and “patient” based on the distinct ways these types of individuals typically communicate. For instance, a doctor's speech may include medical terminology and diagnostic questions, while a patient's speech may consist of symptom descriptions and personal health concerns.

Another approach to diarization may involve utilizing input channels to segment speech data samples. This approach may be used in phone call scenarios, where the audio data is captured separately for each participant. For instance, the audio processing facility 116 may attribute the caller's input to the caller and the agent's input to the call center agent. By using the distinct audio streams from each input channel, the audio processing facility 116 can accurately separate the speech data samples without requiring additional processing to distinguish between speakers.

Another approach to diarization may involve voice prints or vocal signatures to assign roles to speakers. Voice prints may be or include unique acoustic characteristics associated with an individual's voice, such as pitch, tone, and cadence. The audio processing facility 116 may analyze acoustic characteristics to identify and differentiate speakers in the audio data. For example, if a clinical encounter involves a doctor and a patient, the device may use pre-recorded voice samples and/or real-time voice analysis to match a speaker's voice print to their respective role.

Once the speech data samples are assigned to a particular speaker, the diagnostic device 104 can focus the remainder of the analysis on the relevant speaker's samples. For example, in a clinical encounter, the diagnostic device 104 may prioritize the patient's speech data for vocal biomarker analysis while disregarding the doctor's speech. Similarly, in a call center interaction, the diagnostic device 104 may analyze the caller's speech data to assess emotional states such as stress or frustration.

In some embodiments, the audio processing facility 116 combines speech data samples of the prior audio data of prior subjects. Combining samples may involve aggregating individual speech data samples into larger, cohesive units that each satisfy a threshold length. The threshold length may be determined based on the requirements of the machine learning model and/or the nature of the analysis. For instance, the audio processing facility 116 may combine samples to form training data points of approximately 30 to 40 seconds in duration, as this length may be sufficient to capture meaningful vocal biomarkers such as pitch variability, prosody, and pause duration. If the samples are shorter than the threshold length, the audio processing facility 116 may continue to add additional samples until the threshold length is satisfied. For example, if a prior patient's speech data includes three utterances of 10 seconds each, the audio processing facility 116 may combine these utterances to form a single training data point of 30 seconds.

In some embodiments, the audio processing facility 116 utilizes a sliding window approach to combine samples. The sliding window approach may involve creating overlapping speech data samples, where each speech data sample meets the threshold length but includes portions of the previous speech data sample. For example, if the threshold length is 30 seconds and there are 60 seconds of combined speech data samples, the audio processing facility 116 may create a first speech data sample from 0 to 30 seconds, a second speech data sample from 10 to 40 seconds, and so on.

At operation 506, the health record processing facility 118 obtains prior health record data of the one or more prior subjects. The health record processing facility 118 may interface with one or more health record devices 106, which may include EHR systems, hospital information systems, laboratory information management systems, genomic databases, and/or any other clinical data source. The health record processing facility 118 may access the health record devices 106 through standardized APIs, such as those conforming to HL7 FHIR standards, or through proprietary APIs provided by the healthcare institution. In some cases, the health record processing facility 118 may utilize file transfer protocols, direct database queries, or web scraping techniques to extract relevant data when APIs are unavailable or insufficient.

The types of health record data obtained may be broadly categorized into structured and unstructured data. Structured data includes, e.g., discrete, codified information such as demographic details (e.g., age, sex, ethnicity), diagnosis codes (e.g., ICD-10), procedure codes (e.g., CPT), laboratory test results (e.g., LOINC-coded values), medication lists, vital signs, and time-series measurements. The health record processing facility 118 may query specific database tables or fields to retrieve this information, and may filter by subject identifiers, date ranges, or clinical encounter types to improve relevance to the training task.

Unstructured data includes, e.g., free-text clinical notes, physician summaries, discharge reports, imaging narratives, and any other narrative documentation. To obtain this data, the health record processing facility 118 may extract text fields from EHR systems or document management systems. In some implementations, the health record processing facility 118 may perform OCR to digitize handwritten or scanned documents. The health record processing facility 118 may also retrieve associated metadata including, e.g., document timestamps, author information, and document type, to provide additional context for downstream processing.

In addition to or instead of EHR data, the health record processing facility 118 may obtain other health record data, which includes, e.g., genomic data (e.g., single nucleotide polymorphisms, polygenic risk scores), imaging data (e.g., DICOM files from radiology systems), and data from wearable devices or remote monitoring systems. For genomic data, the health record processing facility 118 may interface with laboratory information systems or external genomic databases, retrieving information such as VCFs, genetic test reports, or structured genetic risk scores. For imaging data, the facility may access picture archiving and communication systems (PACS) and retrieve for example image files and radiology reports.

The health record processing facility 118 may handle data from multiple institutions or sources, which may use different data schemas, coding systems, or storage formats. To address these differences, the health record processing facility 118 may perform data normalization and/or harmonization that, e.g., map disparate coding systems to a common ontology (e.g., mapping local diagnosis codes to ICD-10, or medication names to RxNorm). The health record processing facility 118 may also or instead perform data cleaning steps to correct errors, handle missing values, and/or standardize units of measurement.

In some embodiments, the health record processing facility 118 may perform batch data extraction, where the facility periodically retrieves large datasets for offline model training, and/or real-time streaming, where new health record data is ingested as it is created.

At operation 508, the health record processing facility 118 extracts features from the prior health record data. The health record processing facility 118 may perform extraction via, e.g., NLP, structured data parsing, named entity recognition, and/or any other feature engineering techniques.

For structured data, extracting features may include parsing discrete fields from EHRs and related databases. These fields may include demographic information (e.g., age, sex, ethnicity, education level), diagnosis codes (e.g., ICD-10, SNOMED CT), procedure codes (e.g., CPT), laboratory test results (e.g., blood glucose, cholesterol, hemoglobin A1c), medication lists (e.g., drug names, dosages, and administration dates), vital signs (e.g., blood pressure, heart rate, oxygen saturation), and/or time-series measurements (e.g., longitudinal weight or blood pressure trends). The health record processing facility 118 may normalize the features to, e.g., standardize units (e.g., converting all blood glucose measurements to mg/dL), encode categorical variables (e.g., one-hot encoding for diagnosis codes or medication classes), and/or impute missing values using statistical or model-based methods. For certain features, such as time-series data, the health record processing facility 118 may compute summary statistics such as mean, standard deviation, slope, and/or detect trends and anomalies over time windows relevant to the clinical context.

For unstructured data, such as free-text clinical notes, discharge summaries, and imaging narratives, extracting features may include text preprocessing, including tokenization, sentence segmentation, stopword removal, and/or lemmatization or stemming. Extracting features may also involve named entity recognition (NER) models, which may be based on transformer architectures (e.g., BERT, BioBERT, ClinicalBERT), to identify and extract clinically relevant entities such as symptoms, diagnoses, medications, procedures, and family history. Extracting features may also involve utilizing relation extraction models to determine relationships between entities (e.g., linking a medication to an adverse event or a diagnosis to a symptom onset date). The health record processing facility 118 may generate contextual embeddings for each document or entity using pre-trained language models, which may capture semantic information and can be used as input features for downstream models.

In addition to or instead of entity extraction, extracting features may include generating document-level features such as the frequency of specific terms (e.g., mentions of “tremor” or “cognitive decline”), sentiment or affective tone (e.g., using sentiment analysis models), and section-based features (e.g., extracting information specifically from the “Assessment” or “Plan” sections of a note). For imaging narratives, extracting features may include identifying key findings, impression statements, and/or radiology report codes, which the health record processing facility 118 may then encode as features.

When genomic data is available, extracting features may include parsing variant call files (VCFs) or structured genetic reports to identify, e.g., the presence or absence of specific single nucleotide polymorphisms (SNPs), polygenic risk scores, and known pathogenic variants. The health record processing facility 118 may encode this data as binary indicators, risk scores, or categorical variables.

The health record processing facility 118 may also extract features from other modalities, such as wearable device data or remote monitoring systems. For example, daily step counts, sleep duration, heart rate variability, and activity levels can be summarized over relevant time windows and encoded as features.

In some embodiments, feature extraction may include the use of dimensionality reduction techniques (e.g., principal component analysis, t-SNE) to condense high-dimensional data, such as longitudinal lab results or genomic profiles, into lower-dimensional representations. In some embodiments, the health record processing facility 118 may utilize feature selection algorithms to identify the most predictive features for a given health condition, using methods such as mutual information, correlation analysis, or wrapper-based approaches.

The health record processing facility 118 may organize the extracted features into structured vectors or embeddings for integration with features from other modalities (such as vocal biomarkers).

In some embodiments, feature extraction may include prompting an LLM. The prompt may be configured to elicit a direct prediction or assessment from the LLM. For example, the prompt may include “Given the following medical record, what is the likelihood this patient has [target condition]?” or “Does this patient exhibit clinical features consistent with [disease]?” The LLM processes the prompt and generates a response, which may be a probability score, a categorical label, or a natural language explanation. The health record processing facility 118 may then encode the response as an embedding, e.g., by using the output text directly as a feature, by passing the response through an embedding model, or by extracting the penultimate neural layer activations from the LLM as a dense vector representation.

In some embodiments, prompts may include few-shot or zero-shot prompting, where the prompt includes one or more example records and desired outputs to guide the LLM's reasoning. In some embodiments, retrieval-augmented generation (RAG) techniques are utilized, where the LLM is provided with additional context from external knowledge bases, population-level data, and/or health record data. The resulting LLM-generated prediction embedding may be concatenated with other feature vectors (such as those from speech or genomic data) or used as a standalone input to downstream machine learning models.

At operation 510, the diagnostic device 104 combines the speech features and health record features to form training data. Combining the extracted speech features and health record features may involve aligning, normalizing, and/or integrating multimodal feature vectors into unified data structures suitable for machine learning model development. This process may begin with the association of each subject's speech-derived features (e.g., acoustic, prosodic, temporal, respiratory markers) with the corresponding health record features, which may include structured clinical variables, unstructured text-derived embeddings, genomic indicators, and physiological measurements.

For accurate pairing, the diagnostic device 104 may utilize unique subject identifiers and/or encounter identifiers to match each set of speech features with the correct health record features. In scenarios where multiple speech samples and/or health record entries exist for a single subject, the diagnostic device 104 may aggregate features over pre-defined time windows (e.g., averaging features across all speech samples within a clinical visit, or summarizing health record features over a relevant period). Alternatively, the diagnostic device 104 may treat each speech-health record feature pair as a distinct training instance, enabling the model to learn from temporal variations and context-specific data.

Once matched, the diagnostic device 104 may standardize the feature vectors from each modality. Speech features, which may be high-dimensional (e.g., embeddings from models like XVector, HuBERT, or wav2vec), may be normalized using techniques such as z-score normalization or min-max scaling for comparability across subjects and sessions. Health record features, which may include both continuous variables (e.g., lab values, age) and categorical variables (e.g., diagnosis codes, medication classes), may be similarly normalized and encoded. Categorical variables may be encoded using, e.g., one-hot encoding, entity embeddings, or ordinal encoding, depending on the downstream model architecture.

The diagnostic device 104 may concatenate the speech and health record feature vectors to form a composite feature vector for each training instance. In some embodiments, the diagnostic device 104 may apply dimensionality reduction or feature selection techniques before or after concatenation to reduce redundancy and improve model efficiency. For example, principal component analysis (PCA) may be used to condense high-dimensional speech embeddings, while mutual information-based selection may be applied to health record features.

In some embodiments, the diagnostic device 104 may utilize multimodal fusion strategies rather than concatenation. For instance, attention-based fusion layers or transformer-based architectures can be used to learn optimal weighting and interactions between speech and health record features. Alternatively, the diagnostic device 104 may use late fusion approaches, where separate models are trained on each modality and their outputs (e.g., risk scores or class probabilities) are combined using ensemble methods or meta-learners.

For training data labeling, the diagnostic device 104 associates ground truth labels to each composite feature vector, reflecting the known health condition(s) or diagnostic outcomes for the subject. These labels may be binary (e.g., presence or absence of a disease), multiclass (e.g., specific diagnosis categories), or continuous (e.g., severity scores or risk probabilities).

At operation 512, the diagnostic device 104 trains a machine learning model based on the training data. The diagnostic device 104 labels each training data point with a label corresponding to the known condition of the subject. For example, in a clinical encounter, the training data points may be labeled with health conditions such as depression, anxiety, Parkinson's disease, or cardiovascular stress. These labels may serve as ground truth for the machine learning model, allowing it to learn the relationship between the vocal biomarkers present in the audio data, features (e.g., symptoms, test results) in the health record data, and the corresponding condition.

The diagnostic device 104 may train separate machine learning models for different contexts to account for the distinct characteristics of each scenario. For instance, a model trained on audio data from doctor-patient interactions may focus on identifying health conditions such as neurological disorders or respiratory impairments, while a model trained on agent-customer interactions may prioritize emotional states and behavioral patterns.

During training, the diagnostic device 104 may use the labeled training data to optimize the parameters of the machine learning model. Optimization may involve feeding the training data into the model and adjusting its parameters (e.g., weights and biases) to optimize an objective function, such as minimizing the error between the model's predictions and the ground truth labels. For example, if the model predicts that a subject has depression based on their vocal biomarkers and history of treatment for depression, but the ground truth label indicates that the subject has anxiety, the model's parameters are updated (e.g., via backpropagation) to improve its prediction accuracy. The training process may involve multiple iterations, with the diagnostic device 104 continuously refining the model until the model's predictions achieve a threshold level of accuracy.

In some embodiments, training a model to generate a diagnostic prediction may include fine tuning a pre-trained model. Fine tuning may involve taking an existing pre-trained model, which has already been trained on a broad corpus of general data, and adapting the pre-trained model to the specific task of health condition prediction using the training data. The diagnostic device 104 may initialize the model with the pre-trained weights, which encode general knowledge about language, clinical concepts, and/or multimodal relationships. The diagnostic device 104 may then provide the model with labeled training data, where each instance of training data may include integrated speech and health record features paired with ground truth diagnostic labels. During fine tuning, the diagnostic device 104 may update the model's parameters through backpropagation to minimize a task-specific loss function, such as cross-entropy for classification or mean squared error for regression, thereby enabling the model to learn patterns and associations unique to the clinical prediction task.

In some embodiments, the system may include an agentic AI architecture in which a central agentic AI system orchestrates a plurality of autonomous software processes (or “AI agents”), each configured to perform one or more distinct operations within the overall diagnostic workflow. The agentic AI system may be implemented as a software-based orchestration layer, a set of coordinated microservices, or a distributed computing framework, and may be configured to manage the delegation, sequencing, and/or integration of tasks among the AI agents. Each AI agent may be specialized for a particular function, such as audio preprocessing, vocal biomarker extraction, health record parsing, feature selection, model training, or diagnostic inference, and may operate independently or in collaboration with other agents under the direction of the agentic AI system.

The agentic AI system may dynamically allocate resources and schedule operations based on the current data inputs, system state, and/or diagnostic objectives. For example, upon receiving a new batch of audio and health record data, the agentic AI system may assign the audio preprocessing agent to segment and clean the audio, while simultaneously directing a health record extraction agent to parse and encode relevant health record data. Once these agents complete their respective tasks, the orchestration system may trigger a feature fusion agent to combine the extracted features, and subsequently activate a model training or inference agent to generate diagnostic predictions.

Techniques operating according to the principles described herein may be implemented in any suitable manner. Included in the discussion above are a series of flow charts showing the steps and acts of various processes that generate a diagnostic output based on audio data. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally equivalent circuits such as a Digital Signal Processing (DSP) circuit, Field Programmable Gate Array (FPGA), or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one of ordinary skill in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of software. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionalities may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 606 of FIG. 6 described below (i.e., as a portion of a computing device 600) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 1, or one or more computing devices (or one or more processors of one or more computing devices) may be programmed to execute the computer-executable instructions. A computing device or processor may be programmed to execute instructions when the instructions are stored in a manner accessible to the computing device/processor, such as in a local memory (e.g., an on-chip cache or instruction register, a computer-readable storage medium accessible via a bus, a computer-readable storage medium accessible via one or more networks and accessible by the device/processor, etc.). Functional facilities that comprise these computer-executable instructions may be integrated with and direct the operation of a single multi-purpose programmable digital computer apparatus, a coordinated system of two or more multi-purpose computer apparatuses sharing processing power and jointly carrying out the techniques described herein, a single computer apparatus or coordinated system of computer apparatuses (co-located or geographically distributed) dedicated to executing the techniques described herein, one or more Field-Programmable Gate Arrays (FPGAs) for carrying out the techniques described herein, or any other suitable system.

FIG. 6 illustrates one exemplary implementation of a computing device in the form of a computing device 600 that may be used in a system implementing the techniques described herein, although others are possible. It should be appreciated that FIG. 6 is intended neither to be a depiction of necessary components for a computing device to operate in accordance with the principles described herein, nor a comprehensive depiction.

Computing device 600 may comprise at least one processor 602, a network adapter 604, and computer-readable storage media 606. Computing device 600 may be, for example, a desktop or laptop personal computer, a personal digital assistant (PDA), a smart mobile phone, a server, a wireless access point or other networking element, or any other suitable computing device. Network adapter 604 may be any suitable hardware and/or software to enable the computing device 600 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable storage media 606 may be adapted to store data to be processed and/or instructions to be executed by one or more processors 602. Processor 602 enables processing of data and execution of instructions. The data and instructions may be stored on the computer-readable storage media 606.

The data and instructions stored on computer-readable storage media 606 may comprise computer-executable instructions implementing techniques which operate according to the principles described herein. In the example of FIG. 6, computer-readable storage media 606 stores computer-executable instructions implementing various facilities and storing various information as described above. Computer-readable storage media 606 may store the various processes/facilities discussed above. In some embodiments, the diagnostic device 104 is a computing device 600 and the computer-readable storage media 606 may store the audio processing facility 116, health record processing facility 118, and diagnostic facility 120. In some embodiments, the client computing device 102 is a computing device 600 and the computer-readable storage media 606 may store the audio capture facility 126 and the user interface facility 128. In some embodiments and the client computing device 102 are a computing device 600 and the computer-readable storage media 606 may store the audio processing facility 116, health record processing facility 118, diagnostic facility 120, audio capture facility 126, and user interface facility 128.

While not illustrated in FIG. 6, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc., described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the principles described herein. Accordingly, the foregoing description and drawings are by way of example only.

What is claimed is:

1. A method comprising:
determining a prediction of whether a subject has any of one or more health conditions, wherein determining the prediction comprises:
in response to determining that audio data includes a plurality of samples of speech of the subject, determining the prediction based at least in part on an analysis of the plurality of samples of speech and health record data,
wherein the analysis of the plurality of samples of speech and the health record data comprises analyzing the audio data using one or more trained multimodal models, the one or more trained multimodal models having been trained with training data including audio data of speech of prior subjects and health record data regarding health of a plurality of prior subjects and information indicating whether each prior subject of the plurality of prior subjects had one or more health conditions; and
outputting the prediction of whether the subject has any of the one or more health conditions.

2. The method of claim 1, wherein the health record data is health record data of the subject.

3. The method of claim 1, wherein the health record data is health record data regarding a group of people of which the subject is a member.

4. The method of claim 1, wherein the health record data comprises structured data and unstructured data.

5. The method of claim 4, wherein the structured data comprises any one or more of demographic details, diagnosis codes, procedure codes, lab results, medication lists, or time-series measurements.

6. The method of claim 4, wherein the unstructured data comprises any one or more of clinical notes, physician summaries, discharge reports, or narrative documentation.

7. The method of claim 1, wherein the health record data comprises genomic data, and wherein the analysis includes determining a correlation between a vocal biomarker and a genetic risk factor.

8. The method of claim 1, wherein the analysis comprises generating a first score based on the audio data, a second score based on the health record data, and a composite score based on a combination of the first and second scores, and wherein the prediction includes the composite score.

9. The method of claim 1, further comprising outputting, together with the prediction, one or more citations to particular health record data contributing to the prediction.

10. The method of claim 1, wherein analyzing the audio data and the health record data regarding the health of the subject comprises:
extracting vocal biomarkers for the subject from the audio data of the speech of the subject;
extracting information regarding health of the subject from the health record data of the subject; and
determining the prediction based at least in part on an analysis of the vocal biomarkers and the information regarding the health of the subject.

11. The method of claim 10, wherein the analysis of the vocal biomarkers and the information regarding the health of the subject includes embedding the vocal biomarkers to form a plurality of vocal biomarker embeddings and embedding the information regarding the health of the subject to form a plurality of health record feature embeddings.

12. The method of claim 11, wherein the analysis of the vocal biomarkers and the information regarding the health of the subject further includes concatenating at least one of the vocal biomarker embeddings and at least one of the health record feature embeddings.

13. The method of claim 11, wherein the analysis of the vocal biomarkers and the information regarding the health of the subject further includes multimodal fusion of the vocal biomarkers and the information regarding the health of the subject.

14. The method of claim 10, wherein extracting information regarding health of the subject from the health record data comprises providing a focusing prompt to a pre-trained large language model.

15. The method of claim 10, wherein extracting information regarding health of the subject from the health record data comprises obtaining additional context from external knowledge bases by retrieval augmented generation.

16. The method of claim 1, further comprising:
receiving over time one or more segments of audio data, at least one of the segments of the audio data including audio data of speech; and
identifying, from among the one or more segments of audio data, the plurality of samples of speech of the subject.

17. The method of claim 16, wherein determining the prediction of whether the subject has any of one or more health conditions comprises:
iteratively repeating over the time the determining the prediction of whether the subject has the one or more health conditions, wherein in each iteration of the iteratively repeating, the determining the prediction is performed using a different portion of the one or more segments of audio data received over the time, and wherein, in each iteration of the iteratively repeating, the determining the prediction is performed using a different portion of the health record data temporally aligned with the different portion of the one or more segments of audio data.

18. The method of claim 17, wherein, in each iteration of the iteratively repeating, the determining the prediction is performed using a different portion of the health record data temporally aligned with the different portion of the one or more segments of audio data.

19. The method of claim 17, wherein:
the iteratively repeating comprises at least a first iteration and a second iteration;
in the first iteration, the determining the prediction is performed using a first set of segments of audio data, the first set of segments of audio data being fewer than all of the segments of audio data received over the time;
in the second iteration, the determining the prediction is performed using a second set of segments of audio data, the second set of segments of audio data being fewer than all of the segments of audio data received over the time; and
the first and second sets of segments of audio data partially overlap.

20. The method of claim 17, wherein identifying the plurality of samples of speech of the subject from among the segments of audio data comprises:
determining, when a segment of audio data comprises speech, whether the speech is speech of the subject;
in response to determining that a segment of audio data comprises speech of the subject, determining whether at least some audio data of the speech of the subject included within the segment of audio data satisfies one or more criteria for use as a speech sample; and
in response to determining that the at least some audio data of the speech of the subject satisfies the one or more criteria for use as a speech sample, including the at least some audio data as a sample of speech of the subject in the samples of speech of the subject.

21. The method of claim 20, further comprising:
when the audio data comprises audio of speech of multiple speakers, repeating the determining a prediction of whether a subject has any of one or more health conditions for each of at least one other speaker of the multiple speakers, wherein the health record data corresponds to the other speaker.

22. The method of claim 20, wherein determining whether the at least some audio data of the speech satisfies one or more criteria for use as a speech sample comprises determining whether the at least some audio data of the speech has a duration longer than a threshold duration.

23. The method of claim 20, wherein determining whether the at least some audio data of the speech satisfies one or more criteria for use as a speech sample comprises evaluating an acoustic quality of the at least some audio data.

24. The method of claim 1, further comprising:
receiving the audio data, the audio data having been captured during a clinical encounter between the subject and a clinician.

25. The method of claim 1, further comprising:
receiving the health record data, the health record data having been recorded during a clinical encounter between the subject and a clinician.

26. The method of claim 1, further comprising:
receiving the audio data, the audio data having been captured during at least one time the subject was speaking to another person within range of a microphone.

27. The method of claim 1, further comprising:
receiving the health record data, the health record data having been obtained from a remote health data repository.

28. The method of claim 1, wherein the audio data and the health record data are different modalities.

29. At least one computer-readable storage medium storing computer-executable instructions that, when executed by at least one processor, cause the at least one processor to carry out a method comprising:
determining a prediction of whether a subject has any of one or more health conditions, wherein determining the prediction comprises:
in response to determining that audio data includes a plurality of samples of speech of the subject, determining the prediction based at least in part on an analysis of the plurality of samples of speech and health record data,
wherein the analysis of the plurality of samples of speech and the health record data comprises analyzing the audio data using one or more trained multimodal models, the one or more trained multimodal models having been trained with training data including audio data of speech of prior subjects and health record data regarding health of a plurality of prior subjects and information indicating whether each prior subject of the plurality of prior subjects had one or more health conditions; and
outputting the prediction of whether the subject has any of the one or more health conditions.

30. An apparatus comprising:
at least one processor; and
at least one storage medium having stored thereon executable instructions that, when executed by the at least one processor, cause the at least one processor to carry out a method of predicting a health condition in a subject, the method comprising:
determining a prediction of whether a subject has any of one or more health conditions, wherein determining the prediction comprises:
in response to determining that audio data includes a plurality of samples of speech of the subject, determining the prediction based at least in part on an analysis of the plurality of samples of speech and health record data,
wherein the analysis of the plurality of samples of speech and the health record data comprises analyzing the audio data using one or more trained multimodal models, the one or more trained multimodal models having been trained with training data including audio data of speech of prior subjects and health record data regarding health of a plurality of prior subjects and information indicating whether each prior subject of the plurality of prior subjects had one or more health conditions; and
outputting the prediction of whether the subject has any of the one or more health conditions.
